(19) United States
(12) Patent Application Publication
(10) Pub. No.: US 2006/0246531 A1
(43) Pub. Date: Nov. 2, 2006

(54) Chemo-enzymatic process for proteome-wide mapping of post-translational modification

(75) Inventors: Kevan M. Shokat, San Francisco, CA (US); Zachary Knight, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, San Francisco, CA (US)

(21) Appl. No.: 10/539,217

(22) PCT Filed: Dec. 18, 2003
(86) PCT No.: PCT/US03/41118

§ 371 Date: May 2, 2006

Related U.S. Application Data
(60) Provisional application No. 60/434,696, filed on Dec. 18, 2002.

(51) Int. Cl. C12Q 1/37; C07K 14/47

(52) U.S. Cl. 435/23; 530/409

(57) ABSTRACT

The invention provides a method for mapping the location of the post-translational modifications of a post-translationally modified peptide. Also provided is a solid-phase support that includes a reagent for modifying a post-translationally modified amino acid residues of a post-translationally modified, converting it into a substrate for a peptidase.

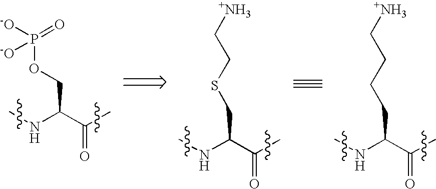

### CROSS REFERENCE TO RELATED APPLICATIONS

This application claims priority to U.S. Provisional Patent Application Ser. No. 60/434,696, filed Dec. 18, 2002, which is herein incorporated by reference in its entirety for all purposes.

### STATEMENT AS TO RIGHTS TO INVENTIONS MADE UNDER FEDERALLY SPONSORED RESEARCH AND DEVELOPMENT

The present invention was made with government support under CA 70031 awarded by the National Institutes of Health (CA 70031). The Government has certain rights to the invention.

### BACKGROUND OF THE INVENTION

Protein phosphorylation is one of the dominant mechanisms of information transfer in cells. A major goal of current proteomic efforts is to generate a system level map describing all the sites of protein phosphorylation. Recent effort toward this goal has focused on developing new technologies for enriching and quantitating phosphopeptides. By contrast, identification of the sites of phosphorylation typically relies exclusively on the use of tandem mass spectrometry to sequence individual peptides.

Much of the complexity of higher organisms is believed to reside in the specific post-translational modification of proteins (Venter et al., Science, 2001, 291(5507): 1304-51.). Protein phosphorylation is the most ubiquitous such modification; almost 2% of the human genome encodes protein kinases and an estimated one-third of all proteins contain a covalently bound phosphate group (Manning et al., Science, 2002, 298(5600): 1912-34). Due to the importance of protein phosphorylation in regulating cellular signaling events, there is intense interest in developing technologies for mapping phosphorylation events on a proteome-wide scale.

Existing approaches for phosphorylation site mapping rely almost exclusively on the use of tandem mass spectrometry (MS/MS) to sequence individual peptides in order to localize sites of phosphorylation. Despite the power of this approach, MS/MS of phosphopeptides remains challenging due to (i) the signal suppression of phosphate containing molecules in the commonly used positive detection mode, (ii) the difficulty in achieving full sequence coverage, especially for long peptides, peptides present in low abundance, and peptides phosphorylated at sub-stoichiometric levels—all of which are common for phosphopeptides, (iii) the difficulty in localizing the phosphoamino acid within an MS/MS spectrum due to the inherent lability of the phosphate group, and (iv) the inability to distinguish between distinct phosphoisoforms of a single polypeptide that may coexist in a biological sample (McLachlin et al., Curr Opin Chem Biol, 2001, 5(5): 591-602; Mann et al., Trends Biotechnol, 2002, 20(6): 261-8; Zhou et al., Nat Biotechnol, 2001, 19(4): 375-8; Oda et al., Nat Biotechnol, 2001, 19(4): 379-82; Steen et al., J Am Soc Mass Spectrom, 2002, 13(8): 996-1003). The challenge of mapping phosphorylation sites is highlighted by recent efforts to enrich phosphopeptides from complex mixtures. While these strategies have provided powerful tools for purifying phosphopeptides, the next step—identifying the precise site of phosphorylation—often fails for many of the peptides that are recovered.

Currently, the first step in mapping the phosphorylation sites of a protein is to digest the phosphoprotein with a protease (e.g., trypsin) that generates smaller peptide fragments for sequencing. We reasoned that this process would be more informative if a protease that specifically cleaved its substrates at the site of phosphorylation were used. Such a digestion would selectively hydrolyze the amide bond adjacent to each phosphorylated residue, facilitating identification of the phosphorylation site directly from the cleavage pattern without sequencing any individual peptide (e.g., from an MS ‘fingerprint’ specifying the exact masses of the cleavage products). Phosphospecific cleavage would also facilitate the interpretation of MS/MS spectra, since the C-terminal residue would always be the formerly phosphorylated residue, resulting in a unique y₁ ion. In this regard, it is often possible to obtain tandem mass spectra of a phosphopeptide, but still fail to localize the phosphoamino acid within that sequence. Presently, no natural protease is known that selectively recognizes a phosphorylated amino acid, or any other post-translational modification.

A method to address this problem utilizing a strategy for specific proteolysis at sites of phosphorylation would represent a significant advance in the art. The present invention satisfies this and other needs.

### BRIEF SUMMARY OF THE INVENTION

In contrast to presently utilized methods of developing a system level map describing all the sites of post-translational peptide modification, e.g., peptide phosphorylation, the present invention provides an approach for post-translational modification mapping that makes it possible to enzymatically interrogate a protein sequence directly to identify sites of post-translational modification.

In a first aspect, the invention provides a method of mapping the site, or plurality of sites, of a post translationally modified peptide. The method includes contacting the peptide with a chemical modification reagent that converts a post-translationally modified amino acid residue of the peptide into a substrate (or a subunit of a substrate) for a peptidase, thereby producing a chemically modified peptide. The chemically modified peptide is contacted with the peptidase under conditions appropriate to degrade the modified peptide, thereby producing a degraded chemically modified peptide, which is subsequently queried to ascertain the locations of post-translational modification.

In an exemplary embodiments, the method further includes, prior to contacting the peptide with a chemical modification reagent, contacting the peptide with an elimination reagent that causes the elimination of a post-translationally added substituent of the post-translationally modified amino acid residue.

In another exemplary embodiment, the method further includes, prior to contacting the peptide with a chemical modification reagent, contacting a substrate amino acid of the peptide that is a natural substrate for the peptidase with a blocking agent thereby converting the substrate amino acid into a side-chain protected amino acid that is not a substrate for the enzyme.

In another exemplary embodiment, the invention provides a method that utilizes the selective chemical transformation of a first phosphorylated amino acid residue into a second amino acid residue that is substantially isosteric with an amino acid residue that is a substrate for a peptidase. Thus, similar to the amino acid residue with which it is substantially isosteric, the second amino acid residue serves as a substrate (or a subunit of a substrate) for the peptidase. The resulting modified polypeptide is then optionally cleaved using a cleaving method that is specific for the second amino acid. The cleaved peptide can then be used to map sites of phosphorylation.

The present invention provides the first example of selective proteolysis at any site of post-translational modification. The method of the invention provides a valuable complement to traditional MS/MS sequencing as a strategy for phosphorylation site mapping.

In a second aspect, the present invention provides a reactive solid phase material. The reactive solid phase material typically contains a solid support and a solid phase reagent immobilized on the solid support. The solid phase reagent is reactive towards the synthetically modified amino acid residue. The synthetically modified amino acid residue is produced by elimination of a post-translationally added substituent of the post-translationally modified peptide.

Other aspects, objects and advantages of the present invention will be apparent from the detailed description that follows.

### BRIEF DESCRIPTION OF THE DRAWINGS

FIG. 1 is a pictorial representation of representative differences between genomics and proteomics.

FIG. 2 is a reaction scheme demonstrating the conversion of phosphoserine to aminoethylcysteine, which can be recognized by lysine specific proteases.

FIG. 3 is a reaction scheme with exemplary conditions that provide a high yielding aminoethylcysteine modification of polypeptides.

FIG. 4 is a schematic representation of two approaches to preparing a tryptic phosphopeptide map. A. Comparison of the cleavage pattern before and after chemical modification gives the identity of phosphorylated residues. B. Reaction with a lysyl/arginyl acylating agent blocks trypsin digestion at those sites; chemical modification then allows single cleavage at the site of phosphorylation.

FIG. 5 displays amino acid residues, their corresponding molecular weights determined by mass spectrometry and HPLC traces of mixtures including the amino acid residue.

FIG. 6 displays amino acid residues and a tabulation of mass spectral data for the residues for an aminoethylcysteine modification and lys-C mapping for diverse phosphoserine peptides.

FIG. 7 displays the phosphorylation sites of the 30 kD phosphoprotein β-casein, which is mapped using aminoethylcysteine modification. Both the cleaved and uncleaved aminoethylcysteine peptides are detected due to α-carbon racemization.

FIG. 8 displays an HPLC chromatogram of peptide containing a cysteic acid residue eluding at approximately 24 minutes (LRRA(cysteic acid)LG) and the digested peptide fragments after cleavage with Asp-N eluting at approximately 20 minutes (LRRA) and approximately 12 minutes ((cysteic acid)LG).

FIG. 9 (A) reaction scheme demonstrating the conversion of phosphothreonine to β-methyl aminoethylcysteine. (B) MALDI-MS spectrum of Lys-C peptidase digested β-methyl aminoethylcysteine modified peptide having the sequence ZFRP(β-methyl aminoethylcysteine) as shown at m/z 698.4, with the undigested β-methyl aminoethylcysteine modified peptide at m/z 1225.5 (having the sequence ZFRP(β-methyl aminoethylcysteine)GFY(Nitro)E) and the undigested phosphorylated peptide ad m/z 1246.5 (having the sequence ZFRPpTGFY(Nitro)E.

FIG. 10 is a time course of trypsin digestion of the phosphoserine peptide (A) and the same peptide modified to contain an aminoethylcysteine residue using the method of the invention (B) as monitored by FRET. Note that the aminoethylcysteine peptide, but not the original phosphoserine peptide, is an efficient trypsin substrate.

FIG. 11 is an HPLC trace showing the separation of the peptide diastereomers of aminoethylcysteine generated using the method of the invention. Monitoring of the course of trypsinization indicates that only one diastereomer (B) is a trypsin substrate, as predicted.

FIG. 12 is a schematic diagram of an overall strategy for phosphorylation site mapping that combines capture, aminoethylcysteine chemical modification and trypsinization steps.

FIG. 13 (A) Mass spectrum of a digested β-casein peptide containing the aminoethylcysteine modification. (B) Mass spectrum of the same digested β-casein peptide containing phosphoserine in place of the aminoethylcysteine modification.

FIG. 14 (A) Mass spectrum at 500 fmol of chemically modified peptide at m/z 1771.9 and 2031.0. (B) Mass spectrum at 250 fmol of chemically modified peptide at m/z 1771.9 and 2031.0. (C) Mass spectrum at 125 fmol of chemically modified peptide at m/z 1771.9 and 2031.0. (D) Mass spectrum at 250 fmol of chemically modified peptide at m/z 1771.9 and 2031.0. (E) Mass spectrum at 25 fmol of chemically modified peptide at m/z 1771.9 and 2031.0.

FIG. 15 is a schematic of a solid-phase method of the invention showing solid phase capture and release.

FIG. 16 is an exemplary synthetic scheme for a resin of use for solid-phase capture.

FIG. 17 is a scheme showing the application of resin for solid-phase capture.

FIG. 18 is a diagram outlining the use of the solid-phase scheme of the invention for the capture and release of aminoethylcysteine peptides.

FIG. 19 displays a tandem mass spectra of aminoethylcysteine modified peptide.

FIG. 20 (A) Scheme for transformation of phosphoserine residues to dehydroalanine, then aminoethylcysteine. a. Sat. Ba(OH)₂:H₂0:DMSO:EtOH:5M NaOH (12:16:12:4:0.5), 1 hour, RT. b. 500 mM cysteamine, 1 hour, RT. (B) HPLC traces of crude reactions cleanly converting phosphoserine peptide 6 (left) to dehydroalanine (middle) then aminoethylcysteine (right).

FIG. 21 (A) Scheme for the capture and modification of phosphoserine peptides using a solid-phase reagent. a. Sat. Ba(OH)₂:H₂0:DMSO:EtOH:5M NaOH (12:16:12:4:0.5), 1 hour, RT, 1 hour, RT. b. 95% TFA, 10 min., RT. (B) Selective capture and modification of phosphoserine peptides using the cysteamine resin. Top, starting material; middle, flow-through; bottom, released aminoethylcysteine peptides.

FIG. 22 (A) Mass spectrum of a peptide containing an aminoethylcysteine modification shown at m/z 1412.8 (NKPPR(aminoethylcysteine)PVVELSK). (B) Mass spectrum of a peptide containing an phosphoserine with no peak at the expected m/z 1433.7 KPPRpSPVVELSK).

FIG. 23 displays a mass spectrum of guanidinated MARCKS peptide after cleavage with Lys-C.

FIG. 24 displays a mass spectrum of an acetylated MARCKS peptide after cleavage with Lys-C.

FIG. 25 displays a mass spectrum of an acetylated MARCKS peptide after cleavage with Lys-C with the detection of the six additional predicted mass peaks.

### DETAILED DESCRIPTION OF THE INVENTION

Definitions

“Peptide” refers to a polymer in which the monomers are amino acids and are joined together through amide bonds, alternatively referred to as a “polypeptide.” The terms “peptide” and “polypeptide” encompass proteins. Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

“Solid support,” as used herein refers to a material that is substantially insoluble in a selected solvent system, or which can be readily separated (e.g., by precipitation) from a selected solvent system in which it is soluble. Solid supports useful in practicing the present invention can include groups that are activated or capable of activation to allow selected species to be bound to the solid support. A solid support can also be a substrate, for example, a chip, wafer or well, onto which an individual, or more than one compound, of the invention is bound.

“Organic functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitrites, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Methods to prepare each of these functional groups are well-known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

A “degraded chemically modified polypeptide” refers to a chemically modified polypeptide where at least one peptide bond had been hydrolyzed by a peptidase.

The term “fragmentation pattern” refers to the configuration of the polypeptide fragments of the degraded chemically modified polypeptide as visualized or produced by an analytical method. A variety of analytical methods may be used to provide a fragmentation pattern. For example, where the analytical method is mass spectrometry, the fragmentation pattern is referred to as a “mass spectral fragmentation pattern.” Where the analytical method is two-dimensional electrophoresis, the fragmentation pattern is referred to as a “two-dimensional electrophoretic fragmentation pattern.”

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

“Solid support,” as used herein refers to a material that is substantially insoluble in a selected solvent system, or which can be readily separated (e.g., by precipitation) from a selected solvent system in which it is soluble. Solid supports useful in practicing the present invention can include groups that are activated or capable of activation to allow selected species to be bound to the solid support. A solid support can also be a substrate, for example, a chip, wafer or well, onto which an individual, or more than one compound, of the invention is bound.

An “substantially isosteric compound” as used herein, is a compound that is sufficiently sterically similar to a natural peptidase substrate such that the compound is recognized as a substrate for the peptidase. For example, β-methyl aminoethylcysteine is substantially isosteric with lysine.

Introduction

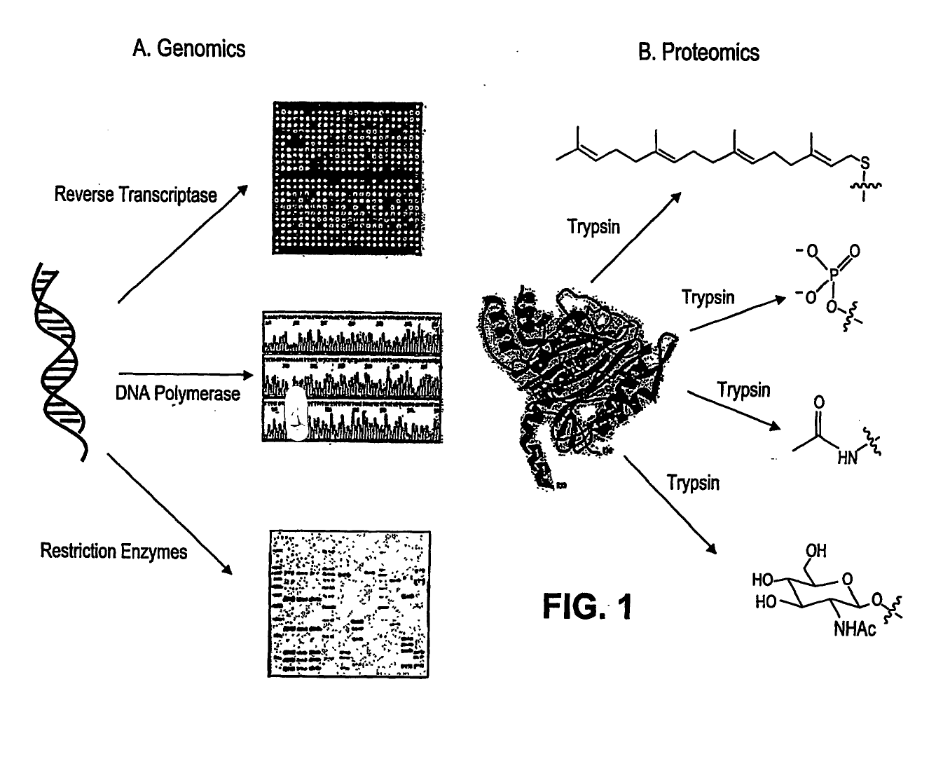

One surprise of the human genome sequence was that there are far fewer genes than many had predicted. Instead, much of the complexity of higher organisms is predicted to reside in the specific modification of proteins, and piecing together this extraordinarily complex web of post-translational modifications is one of the great remaining frontiers in biology (FIG. 1). Phosphorylation is the most ubiquitous and important of these modifications (one-third of all cellular proteins contain covalently bound phosphate), and understanding the molecular logic of protein phosphorylation will be a major step toward decoding biological processes; doing this on a genome wide scale will require new tools that go beyond existing approaches. In view of the importance of phosphorylation, the present invention is illustrated by reference to ascertaining the phosphorylation pattern of a peptide. The focus on phosphorylation is for clarity of illustration and does not limit the scope of the invention.

Mapping Post-Translational Modification Sites

In a first aspect, the invention provides a method of mapping the site, or plurality of sites, of a post translationally modified peptide. The method includes contacting the peptide with a chemical modification reagent that converts a post-translationally modified amino acid residue of the peptide into a substrate (or a subunit of a substrate) for a peptidase, thereby producing a chemically modified peptide. The chemically modified peptide is contacted with the peptidase under conditions appropriate to degrade the modified peptide, thereby producing a degraded chemically modified peptide, which is subsequently queried to ascertain the locations of post-translational modification.

In an exemplary embodiments, the method further includes, prior to contacting the peptide with a chemical modification reagent, contacting the peptide with an elimination reagent that causes the elimination of a post-translationally added substituent of the post-translationally modified amino acid residue.

In another exemplary embodiment, the method further includes, prior to contacting the peptide with a chemical modification reagent, contacting a substrate amino acid of the peptide that is a natural substrate for the peptidase with a blocking agent thereby converting the substrate amino acid into a side-chain protected amino acid that is not a substrate for the enzyme.

Over 300 post-translational modifications are currently known. See the world wide web at URL http://www.abrf.org/index.cfm/dm.home?AvgMass=all, Delta Mass, A Database of Protein Post-Translational Modifications. Exemplary post-translational modifications include phosphorylation, sulfonation, glycosylation, acetylation, methylation, ADP-ribosylation, methionine oxidation, cysteine oxidation, cysteine lipidation, farnesylation, and geranylation. Phosphorylation of specific amino acid residues is the basis for a variety of signaling events that control important cellular processes such as cell growth and differentiation. Typically, post-translational phosphorylation occurs at tyrosine, serine, and threonine residues. Although the current invention is useful in methods of mapping a variety of post translational modifications of amino acids, in one exemplary embodiment, post-translational phosphorylation of serine and threonine residues are mapped using the methods disclosed herein. In another exemplary embodiment, the invention provides a method for ascertaining the location of glycosylated sites on peptides.

Modification Reagents

In some embodiments, the invention provides a two-step chemical process for converting a post-translationally modified amino acid residues in a protein or peptide into the corresponding chemically modified residue. The first step involves contacting a post-translationally modified peptide with an elimination reagent that causes the elimination of a post-translationally added substituent of the post-translationally modified amino acid residue of the peptide. The second step includes contacting the peptide with a chemical modification reagent that converts a post-translationally modified amino acid residue of the peptide into a substrate (or a subunit of a substrate) for a peptidase, thereby producing a chemically modified peptide.

Elimination reagents useful in the current invention include any appropriate reagent capable of eliminating a post translational modification from an amino acid. The resulting amino acid may be referred to herein as a synthetically modified amino acid. The synthetically modified amino acids of the current invention are capable of being converted to a chemically modified amino acid using a chemical modification reagent. The chemically modified amino acid is then recognized by a peptidase, resulting in cleavage of the post-translationally modified peptide at the chemically modified amino acid.

Exemplary elimination reagents include those reagents that remove a post-translational modification via a β-elimination reaction resulting in a synthetically modified amino acid having an alkene moiety. A wide variety of elimination reagents are useful in producing various β-elimination reactions resulting in a synthetically modified amino acid having an alkene moiety. Because the post-translationally added substituent is a leaving group in the β-elimination reaction, the choice of elimination reagents will depend upon the type of post-translational modification. For example, the present strategy can be applied to mapping peptide glycosylation sites as O-glycosylated residues are known to undergo β-elimination under basic conditions (Mega et al., J Biochem (Tokyo), 1990, 107(1): 68-72). Where the post-translational modification is a glycosylated serine or threonine, the leaving group is the carbohydrate alkoxy group. Thus, reagents useful in the preparation of alkenes via hydro-alkoxy-elimination would be useful as an elimination reagent of the present invention (e.g., alkaline reagents such as sodium borohydride, triethylamine in aqueous hydrazine, and the like). In another example, the post-translational modification is a phosphorylated serine or threonine and the leaving group is a phosphate. Thus, reagents useful in the preparation of alkenes via hydro-phosphoester-elimination would be useful as an elimination reagent of the present invention (e.g., ethanedithiol, barium hydroxide, and the like). β-elimination reactions and useful elimination reagents are reviewed in detail in March, ADVANCED ORGANIC CHEMISTRY, 3rd Ed., John Wiley & Sons, New York, 1985. In an exemplary embodiment, the elimination reagent comprises hydroxide moieties. In a related embodiment, the hydroxide concentration is less than 200 mM. In another related embodiment, the hydroxide concentration is 150 mM or less. In another related embodiment, the hydroxide concentration is approximately 150 mM.

The present methods of mapping post-translational modification sites include contacting the peptide with a chemical modification reagent that converts a post-translationally modified amino acid residue of the peptide into a substrate (or a subunit of a substrate) for a peptidase, thereby producing a chemically modified peptide. In an exemplary embodiment, the chemical modification reagent is capable of reacting with the synthetically modified amino acid to form a chemically modified amino acid that is recognized as a substrate by a peptidase. In another embodiment, the chemical modification reagent and/or the synthetically modified amino acid contains a reactive organic functional group for attachment of the chemical modification reagent to the synthetically modified amino acid.

Reactive organic functional groups and classes of reactions useful in practicing the present invention are generally those that are well known in the art of bioconjugate chemistry. Currently favored classes of reactions are those which proceed under relatively mild conditions. These include, but are not limited to nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive organic functional groups include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.
   Where the synthetically modified amino acid contains an alkene moiety, the chemical modification reagent may be added to the synthetically modified amino acid via a Michael addition reaction.

Chemical modification reagents useful in the current invention produce chemically modified amino acids that are recognized by a peptidase. Thus, the chemical modification reagent typically produces a chemically modified amino acid that contains a side chain group that is structurally and/or electronically similar to a natural amino acid side chain group. In an exemplary embodiment, the chemical modification reagent is selected from an inorganic reagent, a substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In a related embodiment, the chemical modification reagent is an unsubstituted heteroalkyl. In a further related embodiment, the chemical modification reagent is an unsubstituted 2-5 membered heteroalkyl. Useful substituted or unsubstituted heteroalkyl chemical modification reagents include aminoalkylthiol reagents, such as cysteamine. In another related embodiment, the chemical modification reagent is an inorganic reagent. In a further related embodiment, the inorganic reagent is a metal sulfate, such as sodium sulfate.

The chemical modification reagent may be selected to produce a chemically modified amino acid that contains a side chain group that is structurally and/or electronically similar to any known natural amino acid side chain group. In an exemplary embodiment, the chemical modification reagent produces a chemically modified amino acid containing a side chain group that is substantially isosteric with the side chain of lysine such that a lysine-specific peptidase cleaves the peptide at the chemically modified amino acid. In another related embodiment, the chemical modification reagent produces a chemically modified amino acid containing a side chain group that is substantially isosteric with the side chain of aspartate such that an aspartate-specific peptidase cleaves the peptide at the chemically modified amino acid.

Thus, in an exemplary embodiment, a method of mapping the site, or plurality of sites, of a post translationally modified peptide is provided. The method includes contacting the post translationally modified peptide with an elimination reagent that causes the elimination of a post-translationally added substituent of the post-translationally modified amino acid residue of the peptide, thereby producing a synthetically modified amino acid residue. The peptide is contacted with a chemical modification reagent that converts a synthetically modified amino acid residue of the peptide into a substrate (or a subunit of a substrate) for a peptidase, thereby producing a chemically modified peptide. The chemically modified peptide is contacted with the peptidase under conditions appropriate to degrade the chemically modified peptide, thereby producing a degraded chemically modified peptide, which is subsequently queried to ascertain the locations of post-translational modification.

In another exemplary embodiment, the elimination and chemical modification reactions are carried out in an optimized mixture of DMSO, water, and ethanol (See Examples and Adamczyk et al., Rapid Commun. Mass Spectrom. 15: 1481-1488 (2001)). In another exemplary embodiment, reaction length and temperature are limited to one hour at room temperature or two hours at 37° C. In another exemplary embodiment, the β-elimination and Michael addition steps are performed consecutively, such that the addition of chemical modification reagent to the basic reaction mixture in the second step reduces the pH of the reaction to approximately 8. In another exemplary embodiment, the Michael addition step is allowed to proceed for up to 6 hours for phosphothreonine peptides.

In some embodiments, the post-translationally modified peptide is subject to gel electrophoresis to separate the peptide from undesired cellular or chemical components. The methods of forming a chemically modified peptide outlined above my be performed prior to gel electrophoresis or while the peptide is within the gel matrix. In addition, the peptide may be contacted with a peptidase while the peptide is within the gel matrix. Thus, chemical modification and digestion may be performed on a gel or gel slice containing the post-translationally modified peptide.

Peptidases

Any peptidase, including both wild-type and mutants can be used to practice the present invention. Peptidases have been found to contain common structural features (see Stawiski et al., Proc. Natl. Acad. Sci., 97: 3954-3958 (2000)). For example, relative to proteins of similar size, peptidases have smaller than average surface areas, smaller radii of gyration, higher Cα densities, are more tightly packed than other proteins, and have fewer helices and more loops. Based on these structural similarities, peptidase function has been predicted with over 86% accuracy from the primary amino acid sequence of peptides (Id.).

Peptidases of the current invention are typically capable of recognizing the chemically modified amino acid of a post-translationally modified peptide. In some embodiments, the peptidase site-specifically cleaves a peptide bond of the post-translationally modified peptide at the chemically modified amino acid of the peptide to produce a degraded chemically modified peptide. After cleavage at the chemically modified amino acid, the site of post-translational modification may be determined.

Site-specific cleavage refers to peptide bond hydrolysis at a preferred site in a peptide. For example, many peptidases cleave the amide backbone of peptides site-specifically at a preferred amino acid residue and/or residues. Peptidases that site-specifically cleave peptides include, for example, chymotrypsin, which site-specifically cleaves at phenylalanine, tryptophan and tyrosine residues; trypsin, which exhibits preferential cleavage at lysine and arginine residues; elastase, which site-specifically cleaves at alanine residues, and subtilisin, which site-specifically cleaves at tyrosine and phenylalanine residues. Similarly, peptidases of the present invention that cleave site-specifically exhibit preferential cleavage at amino acid residues that have been chemically modified. More detailed information regarding known peptidase cleavage sites may be found, for example, in Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); Bouvier et al. Meth. Enzynol. 248: 614 (1995), and Hardy et al., in AMYLOID PROTEIN PRECURSOR IN DEVELOPMENT, AGING, AND ALZHBEMER'S DISEASE, ed. Masters et al. pp. 190-198 (1994).

A wide variety of methods are useful in determining the specificity of site-specific cleavage. For example, a test peptide containing a fluorescent donor-fluorescent quencher pair can be used to measure the kinetics of cleavage by a peptidase. See, for example, Meldal et al., Anal. Biochem. 195:141-7(1991). The cleavage kinetics of a test peptide containing a particular chemically modified amino acid may be measured and subsequently compared to the cleavage kinetics of a series of control peptides. The control peptides typically contain the same amino acid sequence as the test peptide, with the exception that the amino acid containing the chemically modified amino acid in the test peptide is substituted for an unmodified amino acid in the control peptide amino acid sequences. The unmodified amino acid may be a different amino acid that is the natural substrate of the peptidase (e.g. lysine for trypsin), or the same amino acid that has not been chemically modified.

In an exemplary embodiment, a peptidase site-specifically cleaves a peptide at a chemically modified amino acid when the k_{cat}/Kₘ ratio for the chemically modified test peptide is higher than the k_{cat}/Kₘ ratio for a control peptide or a series of control peptides containing the identical sequence with the exception that the control peptide does not contain the chemically modified amino acid or the natural substrate amino acid at the same position as the chemically modified test peptide. In another exemplary embodiment, a peptidase site-specifically cleaves at a chemically modified amino acid when the k_{cat}/Kₘ ratio is at least about 1.1, 1.2, 1.3, 1.4, 1.5, 1.6, 1.7, 1.8, or 1.9 times higher for the test peptide than the k_{cat}/Kₘ ratio for the control peptide(s). In another exemplary embodiment, a peptidase site-specifically cleaves at a chemically modified amino acid when the k_{cat}/Kₘ ratio is at least about 2, 3, 4, 5, 6, 7, 8, 9, or 10 fold higher for the test peptide than the k_{cat}/Kₘ ratio for the control peptide(s).

In another exemplary embodiment, a peptidase site-specifically cleaves a peptide at a chemically modified amino acid when the k_{cat}/Kₘ ratio is approximately the same as a control peptide containing the identical sequence with the exception that the chemically modified amino acid is replaced with natural substrate amino acid. In a related embodiment, a peptidase site-specifically cleaves at a chemically modified amino acid when the k_{cat}/Kₘ ratio is less than 5 times lower than the control peptide containing the natural substrate amino acid. In another related embodiment, a peptidase site-specifically cleaves at a chemically modified amino acid when the k_{cat}/Kₘ ratio is less than 2 times lower than the control peptide containing the natural substrate amino acid. In another related embodiment, a peptidase site-specifically cleaves at a chemically modified amino acid when the K_{cat}/Kₘ ratio is less than 1.9, 1.8, 1.7, 1.6, 1.5, 1.4, 1.3, 1.2, or 1.1 times lower than the control peptide containing the natural substrate amino acid.

The present method includes site-specifically cleaving a chemically modified peptide at a chemically modified amino acid with a peptidase. Typically, a peptidase that cleaves at a chemically modified amino acid hydrolyzes a peptide bond between two adjacent amino acid residues, wherein the peptide bond is within 10 amino acids in either direction of the chemically modified amino acid. For example, where a post-translationally modified peptide contains a chemically modified amino acid that is an aminoethylcysteine, the peptidases of the present invention will site-specifically cleave the peptide at a peptide bond within 10 amino acid residues, in either the N-terminal direction or the C-terminal direction, of the aminoethylcysteine. Thus, site-specific cleavage at a chemically modified amino acid typically refers to cleavage at a peptide bond between two amino acids, wherein the peptide bond is within ten amino acids in either direction of the a chemically modified amino acid.

In an exemplary embodiment, the peptidase site-specifically cleaves a chemically modified peptide at a peptide bond within 1, 2, 3, 4, 5, 6, 7, 8, 9, or 10 amino acids of the a chemically modified amino acid. In another exemplary embodiment, the endopeptidase site-specifically cleaves a chemically modified peptide at a peptide bond between the a chemically modified amino acid and the amino acid immediately C-terminal to the a chemically modified amino acid or the amino acid immediately N-terminal to the a chemically modified amino acid. Thus, the site of cleavage may be at the peptide bond between the chemically modified amino acid and an amino acid adjacent to the post-translationally modified amino acid.

Useful peptidases of the present include a wide array of endopeptidases. Endopeptidases are peptidases that cleave a non-terminal peptide bond of a peptide substrate. In an exemplary embodiment, the peptidase is selected from a serine endopeptidase, a cysteine endopeptidase, an aspartic endopeptidase, and a metalloendopeptidase.

Serine endopeptidases typically fall within the sub-subclass EC 3.4.21 and are structurally related through a common active site structural motif (see Stroud, Sci. Am., 231: 74-88 (1974)). The active site structural motif is commonly referred to as the “catalytic triad,” which includes a specific three-dimensional arrangement of three amino acids: serine, histidine, and aspartate (see Rusell, J. Mol. Biol., 279: 1211-1227 (1998)). The three amino acids act in concert to cleave the peptide bond of a peptide. The catalytic mechanism involves attack of the serine hydroxyl side chain onto the carbonyl moiety of the peptide bond to form a tetrahedral intermediate, followed by general acid catalysis of the intermediate by the aspartate-polarized histidine (see Voet et al., Biochemistry, Second Ed., p. 395 (1995)).

The three-dimensional structure of the catalytic triad is sufficiently similar between the members of the serine endopeptidase family that the serine endopeptidase catalytic triad can be accurately detected from the amino acid sequence alone (see Fischer et al., Protein Sci. 3: 769-788 (1994); Wallace et al., Protein Sci., 5: 1001-1013 (1996); Wallace et al., Protein Sci., 6: 2308-2323 (1997); Rusell, J. Mol. Biol., 279: 1211-1227 (1998)). Methods for determining the presence of the serine endopeptidase catalytic triad typically involve predicting the angles and distances between amino acids in the active site of a protein using computer-based algorithms that analyze the primary structure of the protein. In some methods, the amino acid sequence is additionally considered in determining serine endopeptidase identity (see Rusell, J. Mol. Biol., 279: 1211-1227 (1998)). Although all serine endopeptidases may not share a high degree of amino acid sequence identity, one skilled in the art will recognize common serine endopeptidase structures by analyzing the three dimensional structure of the active site and detecting the presence of the serine/histidine/aspartate catalytic triad. In fact, the three dimensional spatial relationships of the active site of enzymes are often more informative than the one-dimensional primary sequence alone (Rusell, J. Mol. Biol., 279: 1211-1227 (1998)). For example, although trypsin, chymotrypsin and elastase share similar function, three dimensional backbone structure, and catalytic triad structure, only 24 percent of the amino acids are common to all three of these enzymes (see Stroud, Sci. Am., 231: 74-88 (1974)).

In another exemplary embodiment, the peptidase is a cysteine endopeptidase. Common active site structural motifs have been used to successfully identify members of the cysteine endopeptidase family (see Rusell, J. Mol. Biol., 279: 1211-1227 (1998)). Although cysteine endopeptidases lack the serine/histidine/aspartate catalytic triad of the serine endopeptidase family, similarity in the overall tertiary side chain pattern and shape of the active site may be used to identify members of the cysteine endopeptidase family. In a related exemplary embodiment, the cysteine endopeptidase is any enzyme of the sub-subclass EC 3.4.22, which consists of peptidases characterized by having a cysteine residue at the active site and by being irreversibly inhibited by sulfhydryl reagents such as iodoacetate. Mechanistically, in catalyzing the cleavage of a peptide amide bond, cysteine endopeptidases form a covalent intermediate, called an acyl enzyme, that involves a cysteine and a histidine residue in the active site (Cys25 and His159 according to papain numbering, for example).

In another exemplary embodiment, the peptidase is an aspartic endopeptidase of the subclass EC 3.4.23. In contrast to serine and cysteine endopeptidases, catalysis by aspartic proteinases do not involve a covalent intermediate, although a tetrahedral intermediate exists. The nucleophilic attack is achieved by two simultaneous proton transfers: one from a water molecule to the diad of the two carboxyl groups and a second one from the diad to the carbonyl oxygen of the substrate with the concurrent CO—NH bond cleavage. This general acid-base catalysis, sometimes referred to as a “push-pull” mechanism leads to the formation of a non covalent neutral tetrahedral intermediate.

In another exemplary embodiment, the peptidase is a metalloendopeptidase of the subclass EC 3.4.24. Metalloendopeptidases contain a metal, such as zinc, cobalt or nickel which is catalytically active. The catalytic mechanism typically leads to the formation of a non covalent tetrahedral intermediate after the attack of a metal-bound water molecule on the carbonyl group of the scissile bond. This intermediate is further decomposed by transfer of a glutamic acid proton to the leaving group.

In another exemplary embodiment, the peptidase site-specifically cleaves at a lysine amino acid. Thus, in this embodiment, the side chain of the chemically modified amino acid is substantially isosteric with the side chain of lysine such that the chemically modified peptide is cleaved at the chemically modified amino acid by the peptidase. In a related embodiment, the peptidase is selected from the group consisting of endoproteinase Lys-C (Lyc-C), lysyl endopeptidase, trypsin, plasma kallikrein, oligopeptidase B, tryptase, plasmin, acrosin, granzyme A, yapsin 1, peptidyl-Lys metalloendopeptidase, and magnolsyin. In another related embodiment, the peptidase is selected from the group consisting of endoproteinase Lys-C, lysyl endopeptidase and trypsin.

In another exemplary embodiment, the peptidase site-specifically cleaves at an aspartate amino acid. Thus, in this embodiment, the side chain of the chemically modified amino acid is substantially isosteric with the side chain of aspartate such that the chemically modified peptide is cleaved at the chemically modified amino acid by the peptidase. In a related embodiment, the peptidase is selected from the group consisting of peptidyl-aspartate metalloendopeptidase (i.e. Asp-N) and nepenthesin. In another related embodiment, the peptidase is peptidyl-aspartate metalloendopeptidase.

Other representative enzymes with which the present invention can be practiced include, for example, enterokinase, HIV-1 protease, prohormone convertase, interleukin-1b-converting enzyme, adenovirus endopeptidase, cytomegalovirus assemblin, leishmanolysin, β-secretase for amyloid precursor protein, thrombin, renin, angiotensin-converting enzyme, cathepsin-D and a kininogenase.

Furthermore, the method of the invention is of use to cleave at chemically modified residues that are not predicted to be a substrate for specific proteases. Rather, variants of proteases containing appropriate mutations that accommodate the chemically modified residue are readily identified by methods known in the art in addition to those provided herein. Other derivatization schemes, peptidases and combinations thereof are within the scope and spirit of the present invention and will be apparent to those of skill in the art.

Blocking Agents

As mentioned above, in some embodiments the method may include, prior to contacting the peptide with a chemical modification reagent, contacting a substrate amino acid of the peptide that is a natural substrate for the peptidase with a blocking agent thereby converting the substrate amino acid into a side-chain protected amino acid that is not a substrate for the peptidase.

Thus, in an exemplary embodiment, a method of mapping the site, or plurality of sites, of a post translationally modified peptide is provided. The method includes contacting a substrate amino acid of the peptide that is a natural substrate for the peptidase with a blocking agent thereby converting the substrate amino acid into a side-chain protected amino acid that is not a substrate for the enzyme. The post translationally modified peptide is contacted with an elimination reagent that causes the elimination of a post-translationally added substituent of the post-translationally modified amino acid residue of the peptide, thereby producing a synthetically modified amino acid residue. The peptide is contacted with a chemical modification reagent that converts a synthetically modified amino acid residue of the peptide into a substrate (or a subunit of a substrate) for a peptidase, thereby producing a chemically modified peptide. The chemically modified peptide is contacted with the peptidase under conditions appropriate to degrade the modified peptide, thereby producing a degraded chemically modified peptide, which is subsequently queried to ascertain the locations of post-translational modification.

The purpose of the blocking agent is to protect, or otherwise render inactive, the substrate amino acid toward the peptidase. Thus, the blocking agent will typically form a protecting group on the side chain of the substrate amino acid. The term “protecting group” as used herein, refers to any of the groups which are designed to render the substrate amino acid inactive toward the peptidase. More particularly, the protecting groups used herein can be any of those groups described in Greene et al., Protective Groups In Organic Chemistry, 2nd Ed., John Wiley & Sons, New York, N.Y., 1991. The proper selection of protecting groups for a particular side chain group will generally be governed by the chemical reactivity of the side chain group and/or the need to remove the protecting group under mild conditions. A detailed description of amino acid side chain protecting groups can be found, for example, in Bodanszky, Principles of Peptide Synthesis, 2d Ed (1993).

In an exemplary embodiment, the side-chain protected amino acid is a side-chain protected lysine. The amino group of the lysine side chain may be protected with any appropriate protecting group that is known to protect amine groups generally. For example, a blocking agent may be used to transform the lysine side chain amino to a carbamate, an amide, an N-sulfonyl, an N-sulfenyl, an N-nitro, an N-nitroso, an N-oxide, an imine, an N-alkyl amine, an N-aryl amine, an N-phosphinyl, an N-phosphoryl, or an enamine. In a related embodiment, the side-chain protected lysines include Lys(Aloc), Lys(Ac), Lys(Boc), Lys(biotinyl), Lys(2-bromo-Z), Lys(2-chloro-Z), Lys(Dnp), Lys(Fmoc), Lys(For), Lys(Me)₂, Lys(nicatinoyl), Lys(Tfa), Lys(Tos), Lys(Z), Lys(Z)(isopropyl), Lys(Boc)(isopropyl), Lys(dansyl), Lys(Dde), Lys(Me)₃, Lys(Mtt), Lys(palitoyl), Lys(TNM), Lys(acetimidoyl), Lys(2,4,-dichloro-Z), Lys(Me), Lys(p-nitro-Z), Lys(5/6 FAM), Lys(pyrenebutyryl), Lys(guanidinyl), and derivatives thereof (where Fmoc is 9-fluorenylmethyloxycarbonyl; tBu is t-butyl; Trt is trityl; Boc is t-butoxycarbonyl; Z is carbobenzoxy, Dde is [(4,4-dimethyl-2,6-dioxocyclohex-1-ylidine)ethyl]; 5/6-FAM is (5/6-carboxyfluorescein), Aloc is allyloxycarbonyl, Ac is acetyl; Me is methyl, Dansyl is [5-(dimethylamino)naphthalene-1-sulfonyl, Mtt is methyltrotyl; Dnp is dinitrophenyl; Boc is t-butyloxycarbonyl; and Tfa is trifluoroacetyl).

In a related embodiment, the side-chain protected lysine is Lys(Ac) or Lys(guanidinyl). Any appropriate acetylation agent or guanidination agent may be used to form the Lys(Ac) or Lys(guanidinyl), respectively. In a further related embodiment, the guanidination agent is o-methylisourea and the acetylation agent is sulfosuccinimidyl acetate or acetic anhydride.

In another exemplary embodiment, the side-chain protected amino acid is a side-chain protected aspartate. The carboxylate group of the aspartate side chain may be protected with any appropriate protecting group that is known to protect carboxylate groups generally. For example, a blocking agent may be used to transform the aspartate side chain carboxylate to an ester, an amide, an oxalose, an oxazolines, a stannyl ester, or a hydrazide. In a related embodiment, the side-chain protected aspartate includes Asp(OBzl), Asp(OcHex), Asp(OtBu), Asp(OMpe), Asp(Ofm), Asp(Osu), Asp(2-phenyisopropyl ester), Asp(ONp), and derivatives thereof (where OBzl is O-benzyl, OcHex is O-cyclohexyl, OtBU is O-t-butyl, OMpe is 3-methylpent-3-yl ester, OMe is O-methyl, ONp is O-nitrophenyl, Osu is N-hydoxysuccinimide ester, and Ofm is fluoren-9-yl methylester).

A side-chain protected amino acid is not a substrate for the peptidase when the peptidase exhibits decreased cleavage or no detectable cleavage of a peptide bond at the side-chain protected amino acid. In an exemplary embodiment, the rate of cleavage at the side-chain protected amino acid is decreased at least 50 fold over the rate of cleavage at the substrate amino acid. In another exemplary embodiment, the side-chain protected amino acid is decreased at least 100 fold over the rate of cleavage at the substrate amino acid. In another exemplary embodiment, the side-chain protected amino acid is decreased at least 1000 fold over the rate of cleavage at the substrate amino acid. In another exemplary embodiment, the side-chain protected amino acid is decreased at least 10000 fold over the rate of cleavage at the substrate amino acid. In another exemplary embodiment, there is no detectable cleavage at the side-chain protected amino acid.

Querying the Degraded Chemically Modified Peptide

The methods of mapping the location of a site, or plurality of sites, of a post translationally modified peptide include querying the degraded chemically modified peptide.

A variety of methods are useful in determining the site of post-translational modification after cleavage. Typically, the methods involve analyzing the degraded chemically modified peptide produced by cleaving the chemically modified peptide with a peptidase. Exemplary methods include determining the fragmentation pattern of the peptide fragments and comparing the pattern to a known or predicted pattern, determining the size of the peptide fragments, determining the sequence of the peptide fragments produced, and quantitating the amount of peptide fragments produced. A variety of analytical tools may be employed in conjunction with these methods, including, gel electrophoresis (such as single and multi-dimensional electrophoresis), mass spectrometry (including mass spectrometry peptide sequencing techniques), high performance liquid chromatography (HPLC), nuclear magnetic resonance (NMR), capillary gel electrophoresis, affinity chromatography, Edman degradation, high throughput protein chip technology, and the like.

In an exemplary embodiment, the site of post-translational modification is determined by sequencing the peptide fragments produced by cleaving the chemically modified peptide with a peptidase. Sequencing can be accomplished using any suitable technique, such as Edman degradation or mass spectrometry.

In another exemplary embodiment, the site of post-translational modification is determined from the fragmentation pattern of the degraded chemically modified peptide. The fragmentation pattern may be compared to predicted fragmentation patterns of known peptide sequences, thereby identifying the sites of post-translational modifications. Alternatively, the fragmentation pattern may be compared to a plurality of empirically produced fragmentation patterns to determine the site of post-translational modification. After cleavage, fragmentation patterns may be produced by a variety of methods, including, for example, mass spectrometry and two dimensional gel electrophoresis. These and other methods are discussed in more detail in the “Informatics” section below.

Thus, the step of querying the degraded peptide may be conducted using one or more modes of mass spectrometry. In an exemplary embodiment, querying the degraded chemically modified peptide includes mass spectrographic detection of the presence of a chemically modified amino acid residue of the degraded chemically modified peptide. By detecting the presence of the chemically modified amino acid residue, the site of post-translational modification is determined.

Exemplary Methods of Mapping Post-Translational Modifications

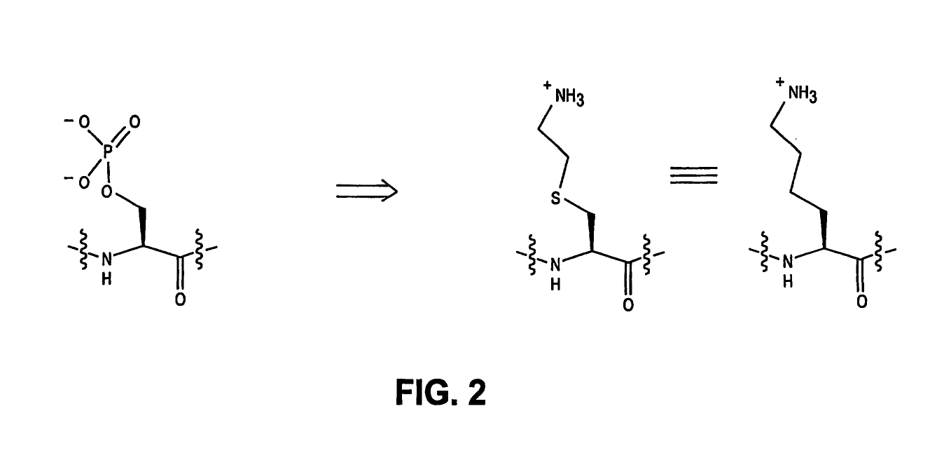

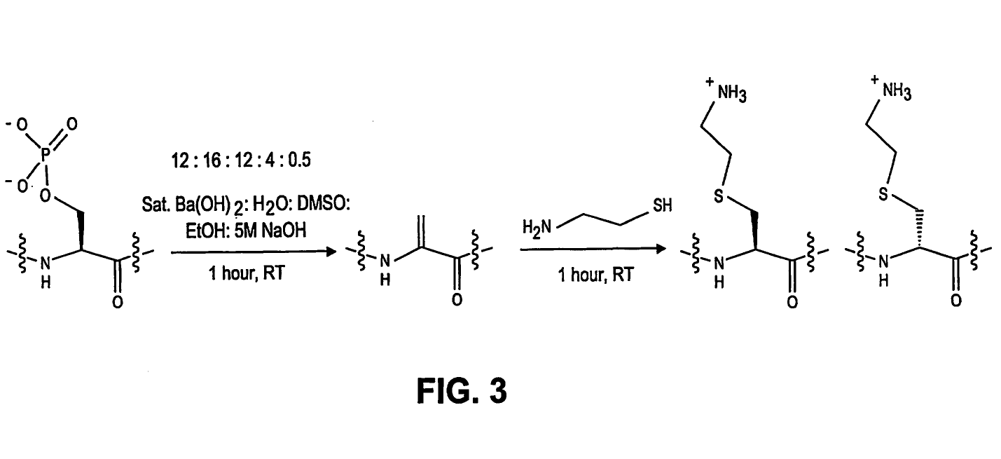

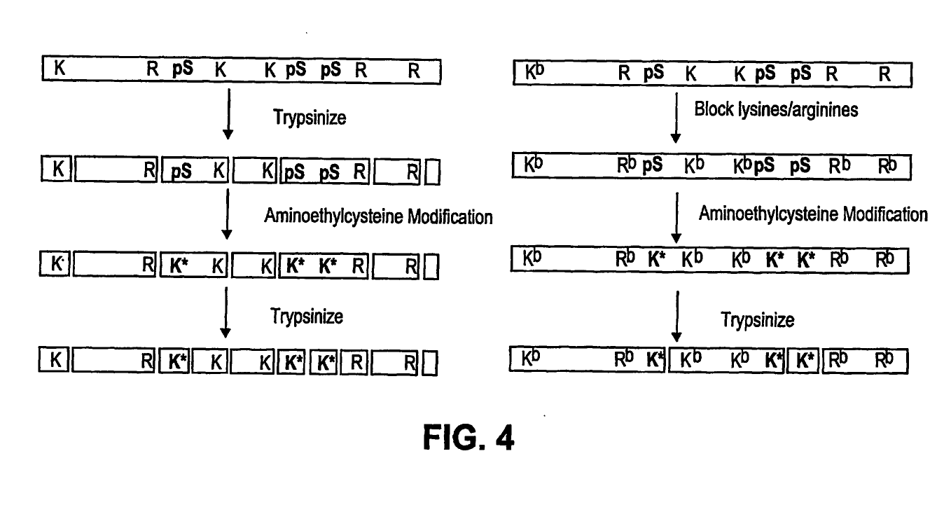

In an exemplary embodiment, the invention provides a two-step chemical transformation for converting phosphoserine residues in proteins or peptides into the corresponding aminoethylcysteine residue (FIG. 2 and FIG. 3). The significance of this transformation is that aminoethylcysteine is isosteric with lysine—the naturally occurring substrate for the lysine specific proteases that are routinely used in protein mapping (e.g. trypsin, lys-C, and lysyl endopeptidase). Since these enzymes cannot distinguish aminoethylcysteine from lysine, digestion of a phosphoprotein that has been subjected to aminoethylcysteine modification with a lysine protease results in peptide cleavage at each phosphorylation site. In this way, it is possible to identify all the serine phosphorylation sites on a protein directly from the masses of each peptide generated in a digest (e.g., from a mass spectrometric fingerprint)—without sequencing any individual peptide (See Table 1 and FIG. 4).

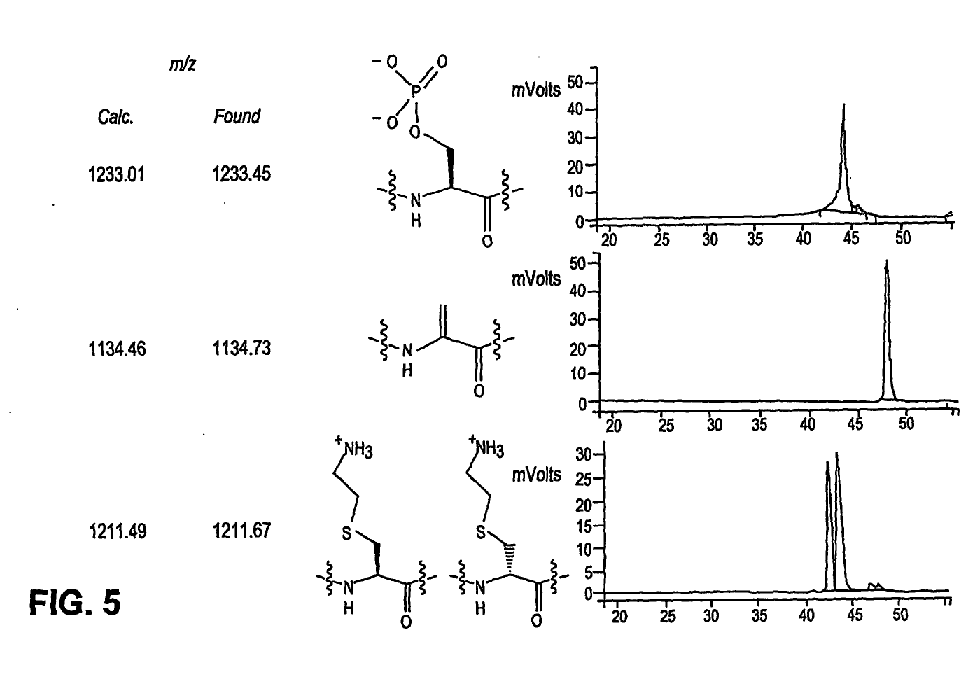

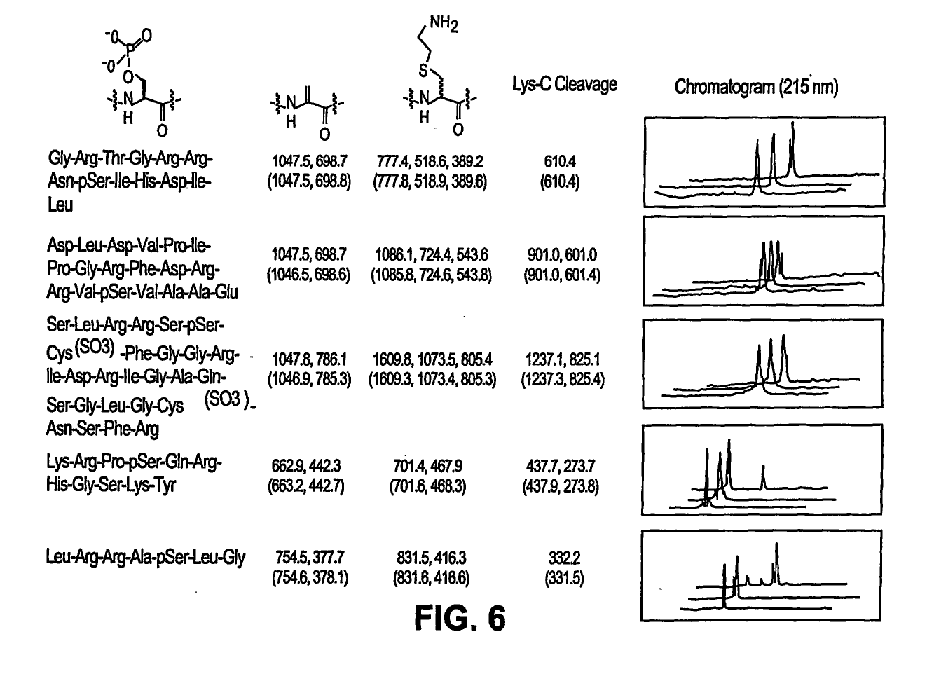

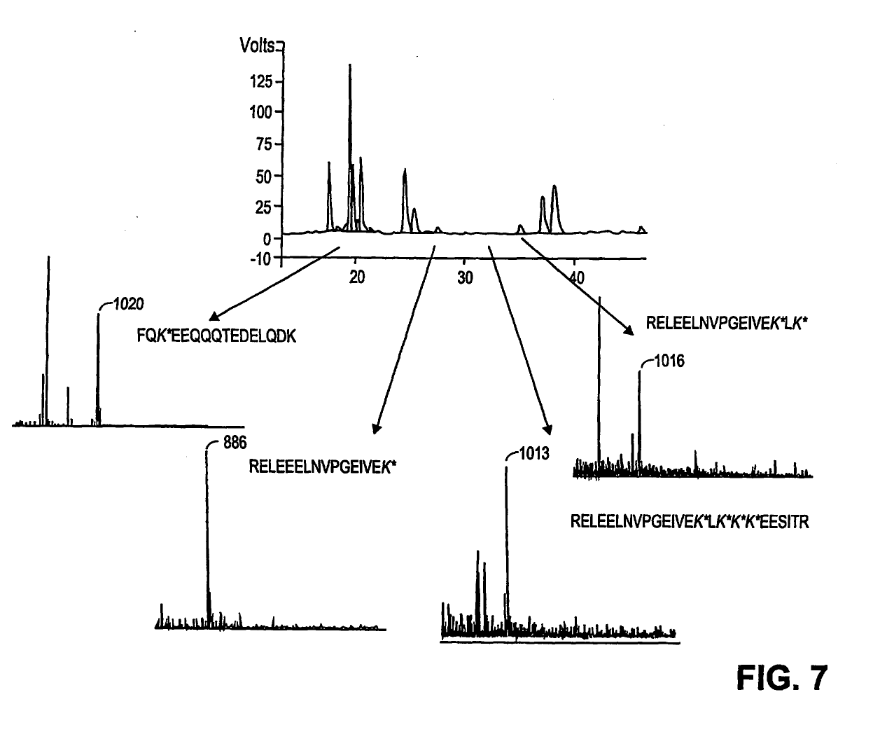

In another exemplary embodiment, in which phosphoserine residues in a peptide are converted into the corresponding aminoethylcysteine residue, the aminoethylcysteine modification chemistry relies in the first step on β-elimination of the phosphate group to form a dehydroalanine intermediate (FIG. 3). In the second step, a cysteamine (in solution or solid phase) adds to the dehydroalanine via a Michael addition reaction. The present invention provides optimized conditions for each reaction such that the chemistry is essentially quantitative (without detectable side-products) and proceeds at room temperature in a single pot in under two hours. The method of the invention works equally well with a wide range of peptides as well as full-length proteins (FIG. 5, FIG. 6 and FIG. 7).

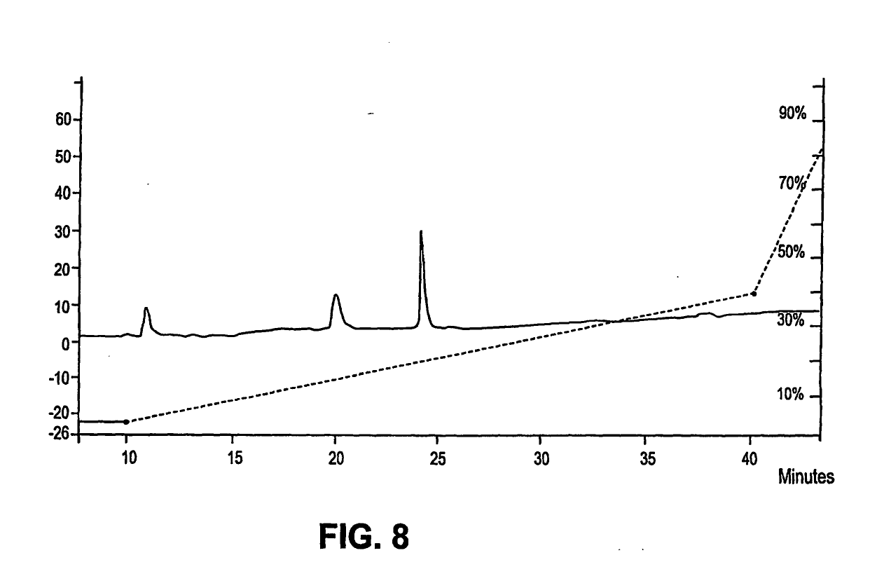

In an exemplary embodiment, the invention provides a two-step chemical transformation for converting phosphoserine residues in proteins or peptides into the corresponding cysteic acid residue (FIG. 8). The β-elimination of the phosphate group forms a dehydroalanine intermediate, which is reacted with a metal sulfonate via a Michael addition reaction to form the corresponding cysteic acid residue. The cysteic acid is an efficient substrate for Asp-N, generating peptide fragments corresponding to specific cleavage at the former site of serine phosphorylation.

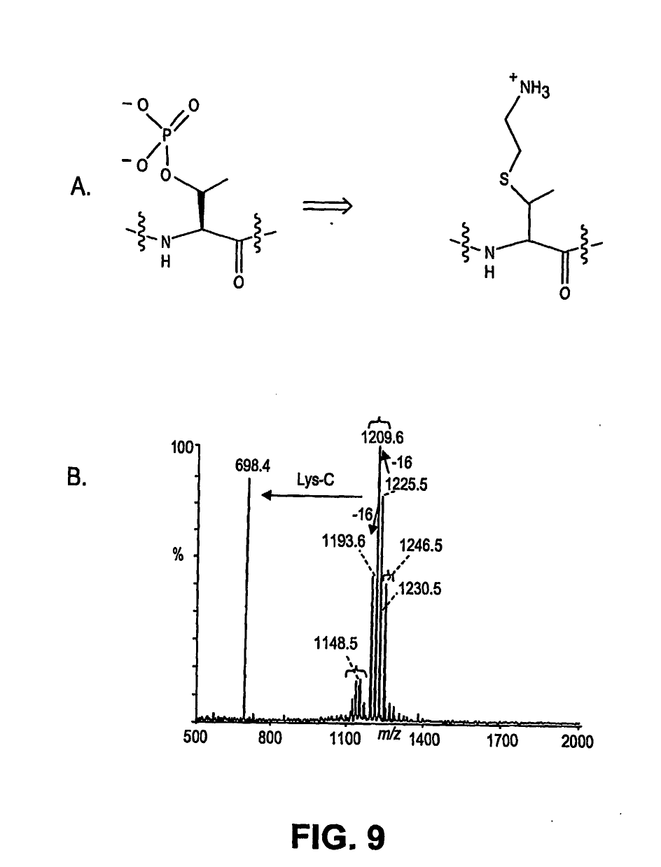

In another exemplary embodiment, the invention provides a two-step chemical transformation for converting phosphothreonine residues in proteins or peptides into the corresponding β-methyl aminoethylcysteine residue (FIG. 9). Again, the β-elimination of the phosphate group forms an alkene intermediate, which is reacted with a cysteamine via a Michael addition reaction to form the corresponding β-methyl aminoethylcysteine. Surprisingly, the β-methyl aminoethylcysteine is an efficient substrate for Lys-C and lysyl endopeptidase, generating peptide fragments corresponding to specific cleavage at the former site of threonine phosphorylation (see Table 1 and FIG. 9).

Exemplary Syntheses of Chemically Modified Peptides

The chemically modified peptides of present invention can be prepared using readily available starting materials or known intermediates in accordance with the teachings below.

In an exemplary embodiment, chemically modified peptides are provided using a two-step chemical process starting with a postranslationally modified peptide containing a post-translationally modified amino acid, as shown in Scheme I below.

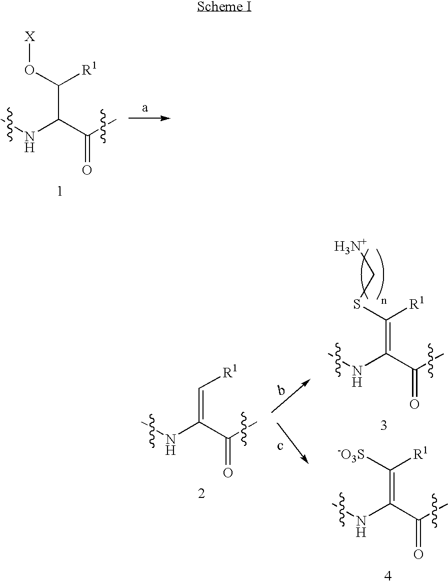

In Scheme I, X is a post-translationally added group selected from phosphoryl, sulfonyl, glycosyl, acetyl, methyl, ADP-ribosyl, lipidyl, farnesyl, and geranyl. In an exemplary embodiment, the post-translationally added group is selected from phosphoryl and glycosyl. In another exemplary embodiment, the post-translationally added group is phosphoryl. R¹ is selected from hydrogen and substituted or unsubstituted alkyl. In an exemplary embodiment, R¹ is selected from hydrogen and unsubstituted C₁-C₆ alkyl. In another exemplary embodiment, R¹ is selected from hydrogen and methyl. The symbol n represents an integer from 1 to 6. In an exemplary embodiment, n is 2.

The post-translationally modified peptide 1 is contacted with an elimination reagent in step a to form the synthetically modified amino acid 2 containing a reactive alkene moiety. In this example, the elimination reagent is barium hydroxide, although one skilled in the art will immediately recognize that any appropriate elimination reagent may be used.

The intermediate 2 may then by diversified with various chemical modification reagents as exemplified in steps b and c above. In step b, an aminoalkylthiol is used to produce the chemically modified amino acid 3. In an exemplary embodiment, a cysteamine chemical modification reagent is used to produce the corresponding aminoethylcysteine or β-methyl aminoethylcysteine. Alternatively, in step c, 2 is transformed to the sulfonyl amino acid 4 using a sulfonation reagent such as a metal sulfonate. In an exemplary embodiment, the chemical modification reagent is sodium sulfonate.

Racemization of the α Carbon of the Amino Acid

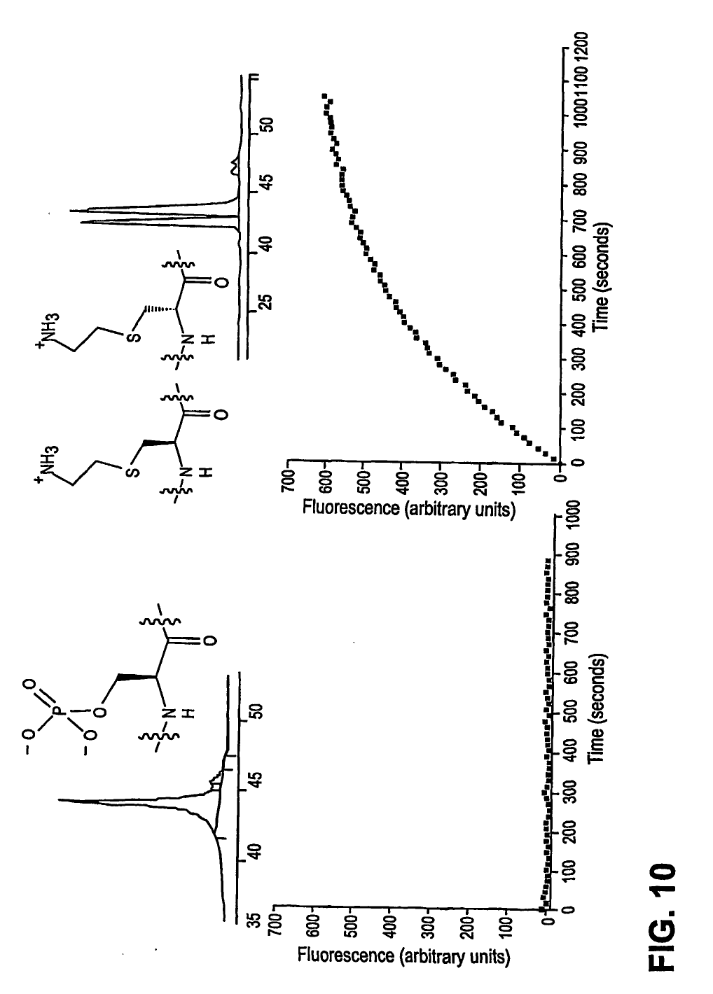

In a further exemplary embodiment, the method of the invention results in the racemization of the α carbon of the amino acid. The utility of this feature of the invention is illustrated by reference to the aminoethylcysteine chemical derivatization and resulting racemization at the α carbon of a phosphorylated amino acid (FIG. 10). The focus of this discussion is for clarity of illustration and should not be construed as limiting the invention.

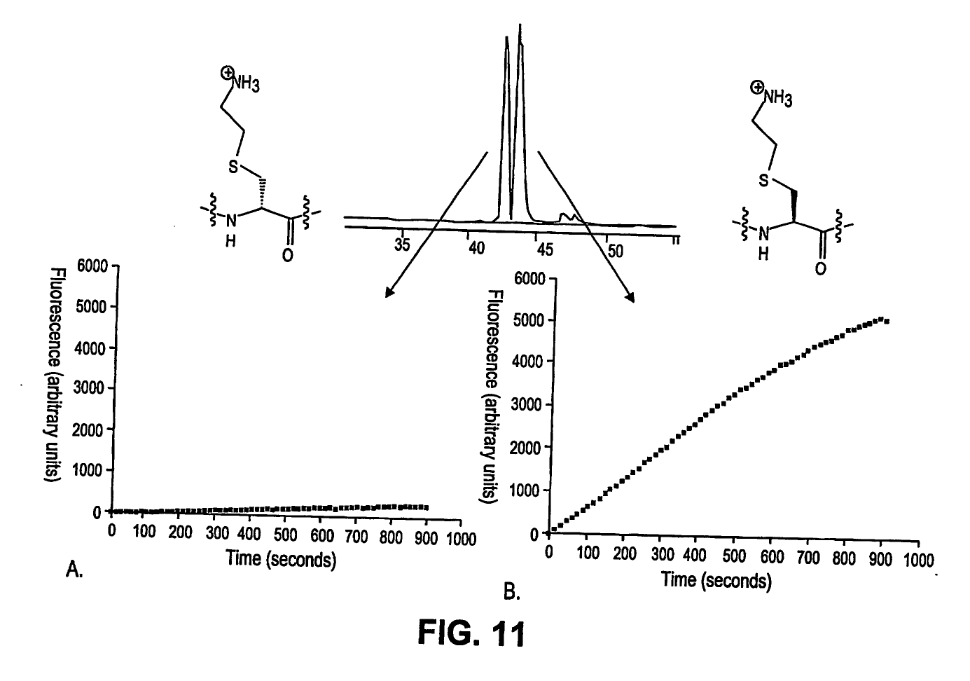

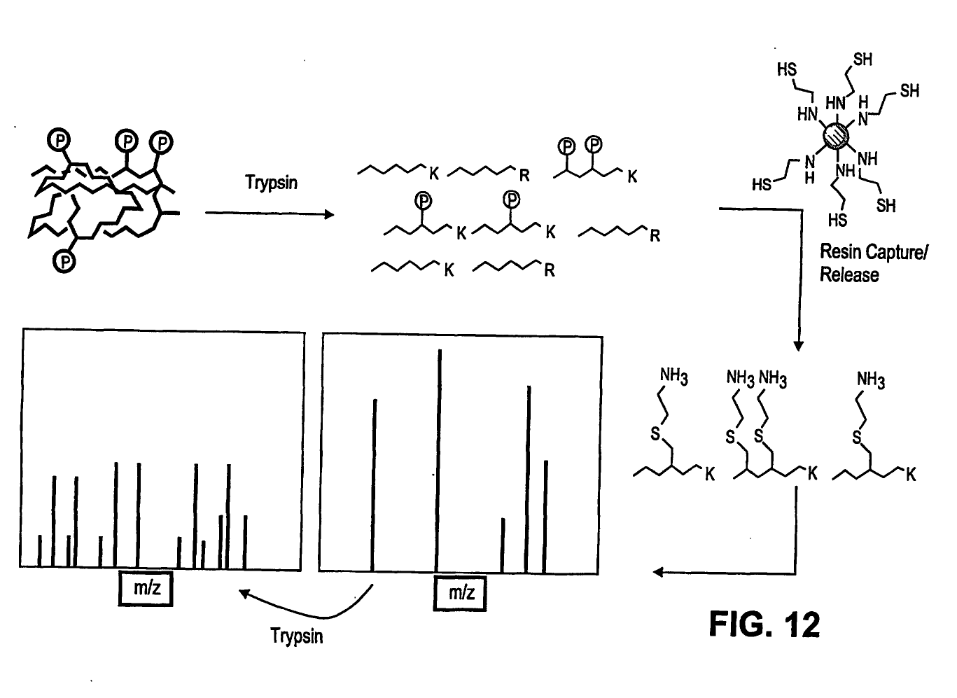

The racemization of the α carbon of the amino acid generates two diastereomeric aminoethylcysteine peptides in an approximately 1:1 mixture. One of these peptides contains the physiological S stereochemistry at the α carbon, and therefore will be a substrate for trypsin-like proteases (FIG. 11). The other peptide contains the non-physiological R stereochemistry, and therefore will not be recognized by trypsin-like proteases. This stereochemical scrambling has an important advantage for phosphopeptide mapping. Under conditions where the proteolytic digestion is allowed to proceed to completion, cleavage will occur at precisely 50% of the sites for any given phosphopeptide. The resultant mass spectrum thus contains peaks for both the intact (derivatized) phosphopeptide, as well as the fragments generated from the cleavage at the phosphorylation site—greatly simplifying database searching to identify the correct peptide (FIG. 7 and FIG. 12). Surprisingly, peptides containing the non-physiological R stereochemistry are efficient substrates for Lyc-C and lysyl endopeptidase (FIG. 9).

Altering the Charge on a Post-Translationally Modified Peptide

The present invention also provides a method for altering the charge on a post-translationally-modified peptide using a reaction such as that set forth herein. It is well established that negatively charged post-translational modification groups generate atypically low signals on the mass spectrometer due to their poor ionization in the positive mode conditions typically used for mass spectrometry, such as electrospray MS and MALDI (matrix assisted laser desorption/ionization) MS. Chemical modifications that result in an amino acid side chain containing a group that is ionizable in the positive mode is beneficial in methods of mapping sites of post-translational modifications where querying the degraded chemically modified peptide is conducted using one or more modes of mass spectrometry.

In an exemplary embodiment, the chemical modification group contains an amino group that is ionizable in the positive mode conditions typically used for mass spectrometry. In a related embodiment, any appropriate chemical modification group containing an amino group is useful in the current methods. Typically, the chemical modification group will additionally contain a reactive organic functional group useful in attaching the chemical modification group to the amino acid.

In a related embodiment, an aminoalkylthiol, such as cysteamine, is used as a chemical modification reagent. For example, cysteamine modification replaces an acidic phosphoserine (two negative charges at pH 7) with a basic aminoethylcysteine (one positive charge at pH 7).

In an exemplary embodiment, the chemical modification increases the sensitivity of post-translational modification detection by at least one order of magnitude. In another exemplary embodiment, the chemical modification increases the sensitivity of post-translational modification detection by at least two orders of magnitude. In another exemplary embodiment, the chemical modification increases the sensitivity of post-translational modification detection by at least 3, 4, 5, 6, 7, 8, 9, or 10 orders of magnitude.

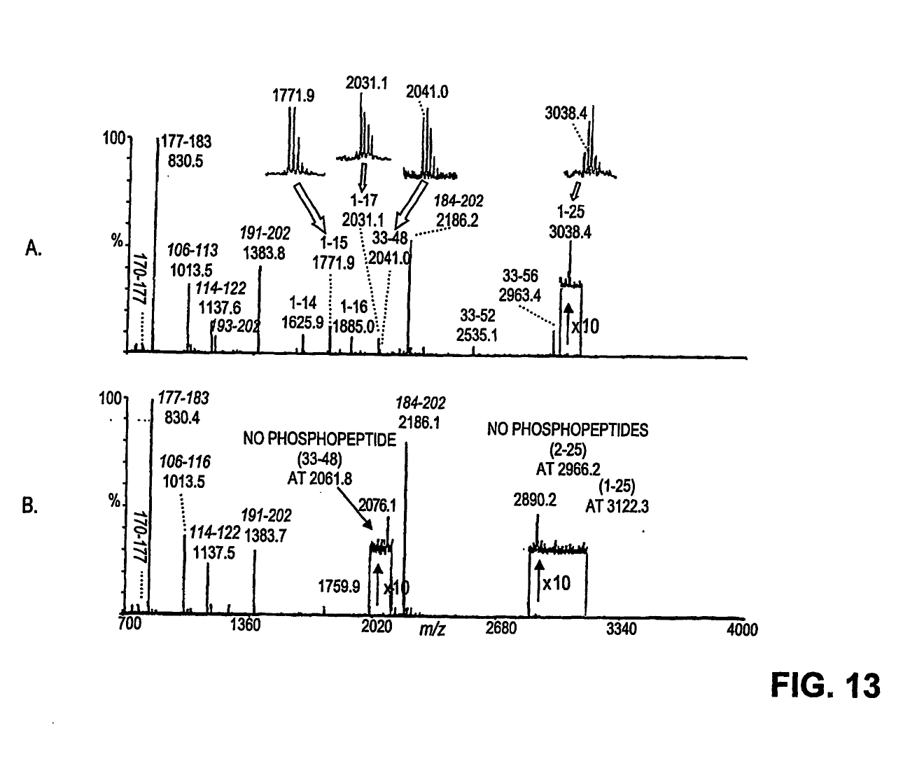

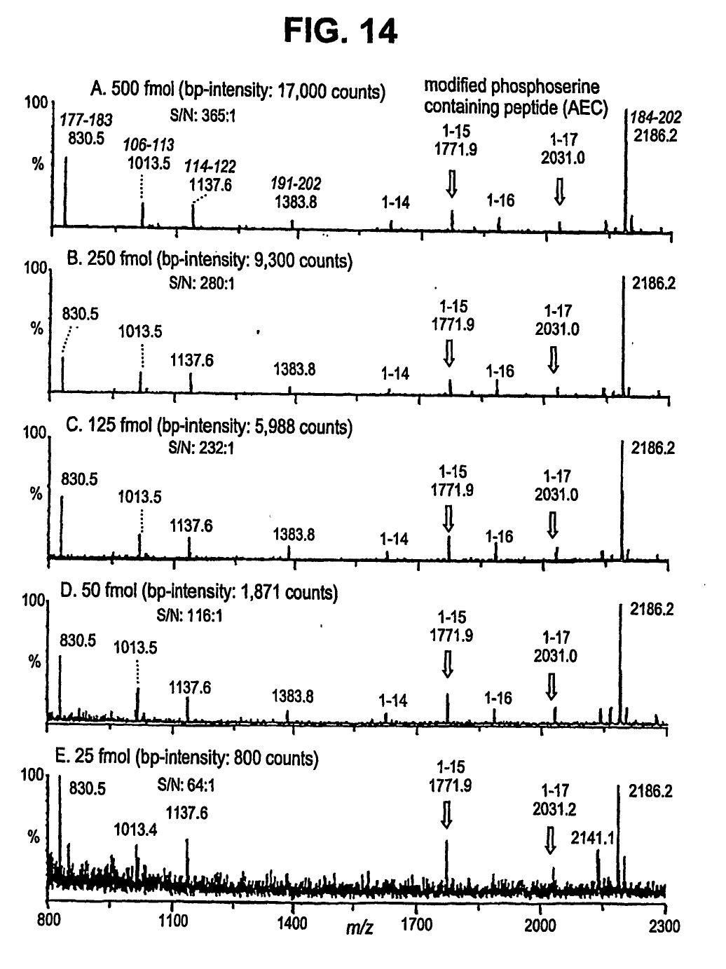

In another exemplary embodiment, the chemically modified amino acid residue of the degraded chemically modified peptide is capable of being detected at a level of 500 fmol or less by MALDI-MS (see Example 1, FIG. 13 and FIG. 14). In another exemplary embodiment, the chemically modified amino acid residue of the degraded chemically modified peptide is capable of being detected at a level of 250 fmol or less by MALDI-MS. In another exemplary embodiment, the chemically modified amino acid residue of the degraded chemically modified peptide is capable of being detected at a level of 125 fmol or less by MALDI-MS. In another exemplary embodiment, the chemically modified amino acid residue of the degraded chemically modified peptide is capable of being detected at a level of 50 fmol or less by MALDI-MS. In another exemplary embodiment, the chemically modified amino acid residue of the degraded chemically modified peptide is capable of being detected at a level of 25 fmol or less by MALDI-MS.

Solid-Phase Methodology and Supports

In another exemplary embodiment, the present invention provides a solid-phase based method for mapping the sites of post-translational modification of peptides. The method includes contacting the post-translationally modified peptide with a solid-phase that is derivatized with solid phase reagent that converts a post-translationally modified amino acid (or an analogue thereof produced by the elimination of a post-translationally added substituent) into a substrate (or subunit of a substrate) for a peptidase.

In another exemplary embodiment, the present invention provides a reactive solid phase material. The reactive solid phase material typically contains a solid support and a solid phase reagent immobilized on the solid support. The solid phase reagent is reactive towards the synthetically modified amino acid residue. The synthetically modified amino acid residue is produced by elimination of a post-translationally added substituent of the post-translationally modified peptide.

In another exemplary embodiment, the present invention provides a method of immobilizing a post-translationally modified peptide comprising a post-translationally modified amino acid. The method includes contacting the peptide with an elimination reagent that causes the elimination of a post-translationally added substituent of the post-translationally modified amino acid residue thereby producing a synthetically modified amino acid. The synthetically modified amino acid is reacted with a solid phase reagent thereby immobilizing the post-translationally modified peptide. The reactive solid phase material typically contains a solid support and a solid phase reagent immobilized on the solid support. The solid support reactive moiety is reactive towards the synthetically modified amino acid residue.

Synthesis on solid supports, “solid-phase synthesis,” is of recognized utility in the synthesis of small molecules, oligomeric compounds and polymers. A diverse array of solid supports bearing useful probes, labels and reactive groups are known in the art (see, for example, Burgess, ed., SOLID-PHASE ORGANIC SYNTHESIS, John Wiley and Sons, 2000; and Chan and White, eds., FMOC SOLID PHASE PEPTIDE SYNTHESIS: A PRACTICAL APPROACH (The Practical Approach Series), Oxford University Press, 2000). Solid supports of use in practicing the present invention include substantially any oligomeric or polymeric material upon which the disclosed synthesis can be performed, and the materials and methods of the present invention are not limited by the identity of the material serving as the solid support. Suitable solid supports for immobilization of a post-translationally modifies peptides (or analogs thereof such as a synthetically modifies peptide) include polymolecular assemblies such as synthetic polymeric resins and gels. Exemplary synthetic polymeric resins and gels include those composed, at least in part, of polystyrene and polyacrylic acids, amides, and esters; glass; polyols such as polyvinyl alcohol and polysaccharides such as agarose, cellulose, dextrans, ficols, heparin, glycogen, amylopectin, mannan, inulin, and starch.

The solid supports of use in the invention typically have a solid phase reagent immobilized thereon. The solid phase reagent includes a reactive organic functional group that is of complementary reactivity to a post-translationally modified amino acid, a synthetically modified amino acid, or analogues thereof.

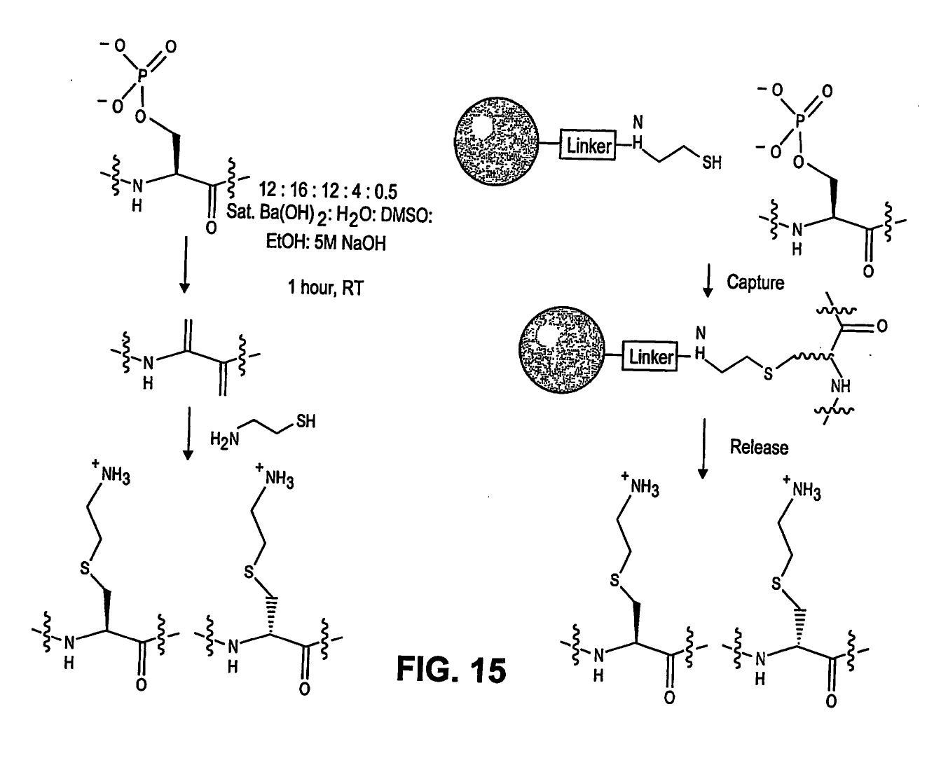

In an exemplary embodiment, the solid phase reagent is equivalent in reactivity to the chemical modification reagents discussed above. In a related embodiment, the synthetically modified amino acid is contacted with the solid phase reagent thereby immobilizing the post-translationally modified peptide. The invention may provide a solid-phase material that includes an equivalent of cysteamine for the Michael addition. Utilizing the material of the invention, it is possible to capture a phosphopeptide or phosphoprotein simultaneous with aminoethylthiol modification (FIG. 15), making it possible to effect phosphopeptide purification and derivatization in one step (FIG. 9).

In some embodiments, the immobilized post-translationally modified peptide is released from the solid support to form a chemically modified amino acid. Any appropriate method may be used to release the immobilized peptide. In an exemplary embodiment, the immobilized post-translationally modified peptide is released from the solid support using a chemical modification reagent, thereby simultaneously releasing the immobilized peptide and forming a chemically modified amino acid as described above.

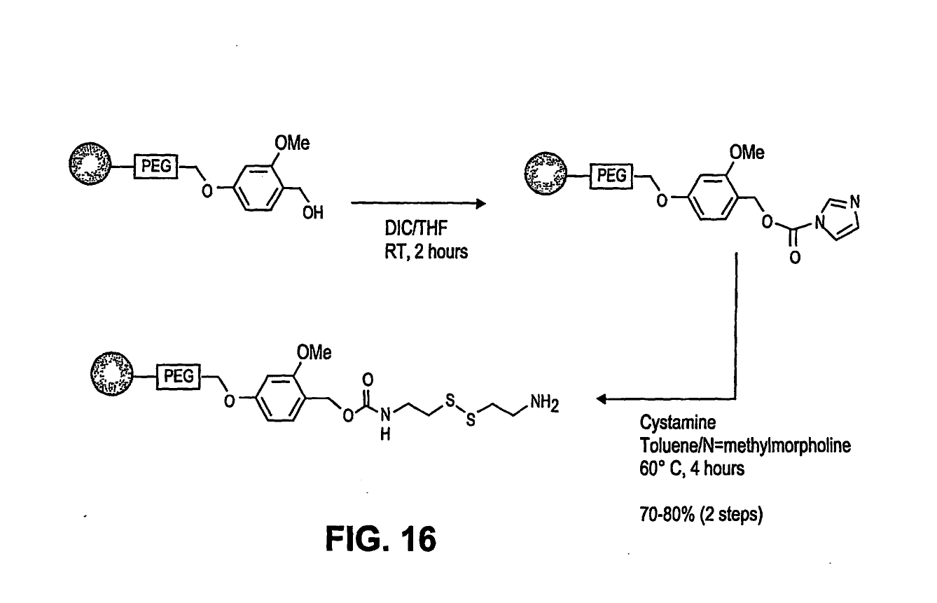

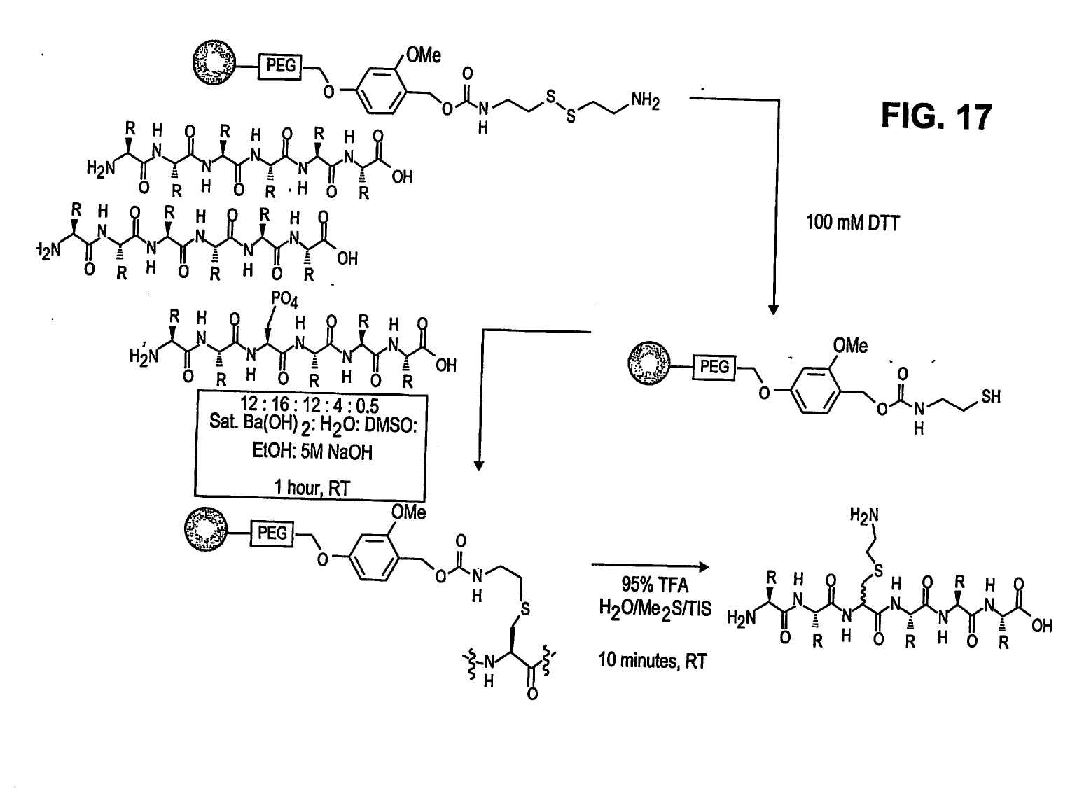

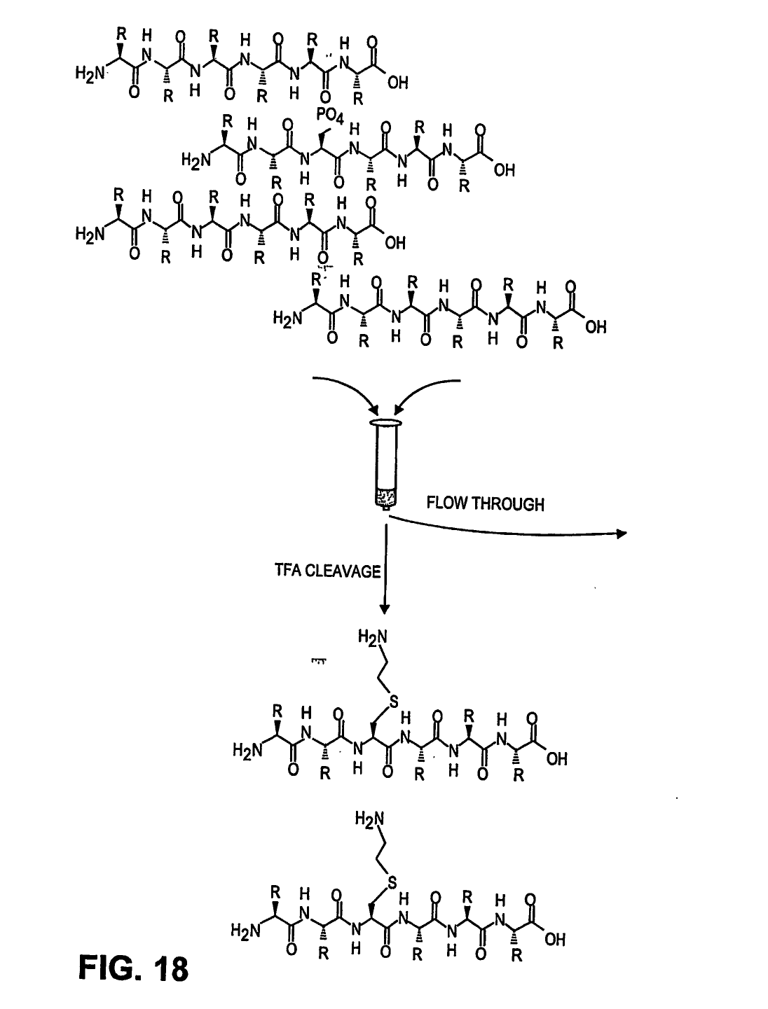

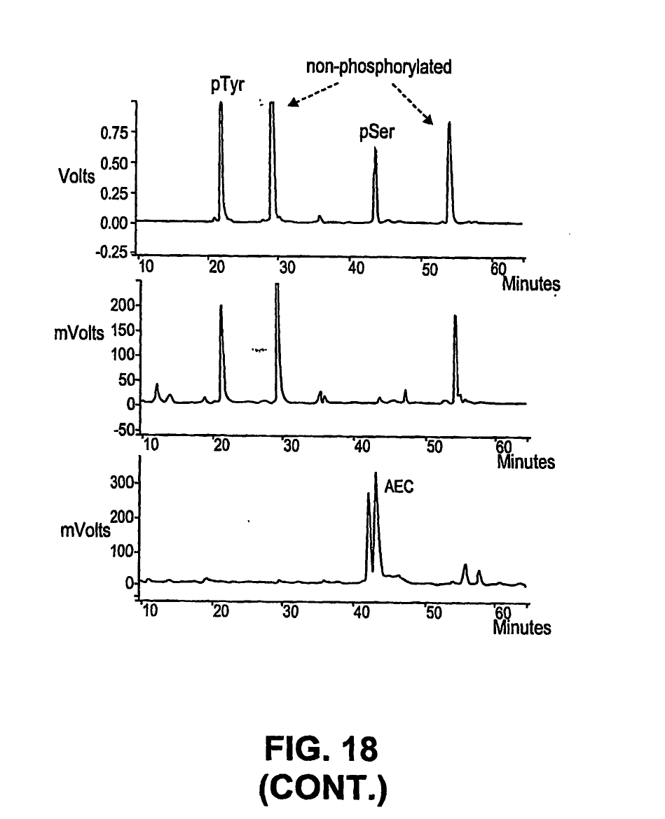

An exemplary solid-phase material of the invention is based on a robust, high-loading resin for this chemistry (FIG. 16 and FIG. 17). The exemplary solid matrix uses a Tentagel base resin that is derivatized to contain a disulfide protected cysteamine linked to the resin through an acid labile carbamate. The resin is useful to purify phosphoserine peptides from contaminating non-phosphorylated and threonine phosphorylated peptides concommitant with derivatization. This resin can be stored indefinitely at −20° C. and deprotected before use. The present invention also provides a method of using the resin to map the post-translational modification, e.g., phosphorylation of a peptide. (FIG. 18).

Informatics

As high-resolution, high-sensitivity datasets acquired using the methods of the invention become available to the art, significant progress in the areas of diagnostics, therapeutics, drug development, biosensor development, and other related areas will occur. For example, disease markers can be identified and utilized for better confirmation of a disease condition or stage (see, U.S. Pat. Nos. 5,672,480; 5,599,677; 5,939,533; and 5,710,007). Subcellular toxicological information can be generated to better direct drug structure and activity correlation (see, Anderson, L., “Pharmaceutical Proteomics: Targets, Mechanism, and Function,” paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see, U.S. Pat. No. 5,811,231). Similar advantages accrue from datasets relevant to other biomolecules and bioactive agents (e.g., nucleic acids, saccharides, lipids, drugs, and the like).

Thus, in an exemplary embodiment, the present invention provides a database that includes at least one set of data assay data. The data contained in the database is acquired using a method of the invention. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases, which include peptide sequence specificity data is for clarity of illustration only. It will be apparent to those of skill in the art that similar databases can be assembled for any assay data acquired using an assay of the invention.

The compositions and methods described herein for identifying and/or quantitating the relative and/or absolute abundance of a variety of molecular and macromolecular species from a biological sample provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

The present invention provides a computer database comprising a computer and software for storing in computer-retrievable form assay data records cross-tabulated, for example, with data specifying the source of the target-containing sample from which each sequence specificity record was obtained.

In an exemplary embodiment, at least one of the sources of target-containing sample is from a tissue sample known to be free of pathological disorders. In a variation, at least one of the sources is a known pathological tissue specimen, for example, a neoplastic lesion or a tissue specimen containing a pathogen such as a virus, bacteria or the like. In another variation, the assay records cross-tabulate one or more of the following parameters for each target species in a sample: (1) a unique identification code, which can include, for example, a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a post-transitionally modified peptide, the invention preferably provides a method for identifying related peptide sequences, comprising performing a computerized comparison between a peptide sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide sequence in a pool of sequences determined from a peptide sample.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, Mv, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In an exemplary embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected binding functionality) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

Kits

The present invention also provides a kit for practicing a method set forth herein. In an exemplary embodiment, the kit includes one or more component useful to practice the method of the invention and instructions for using that component to practice the method of the invention.

In a preferred embodiment, the kit includes a container of a reactive solid support of the invention and instructions for using the solid support to convert a post-translationally modified amino acid residue of a peptide into a substrate for a peptidase. Another exemplary kit further includes a container of the peptidase for which the converted amino acid is a substrate.

The examples that follow are intended to further illustrate the invention not to limit the scope of the invention.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the peptidases described in the peptidase section are equally applicable to the informatics methods or kits described herein. All publications, patents, and patent applications cited herein are hereby incorporated by reference in their entirety for all purposes.

### EXAMPLES

Materials

Sequencing grade Trypsin and Lys-C were from Roche Diagnostics. Tentagel AC resin was from Advanced Chemtech. All peptides were from Anaspec or synthesized using standard Fmoc solid-phase chemistry. All other reagents were from Sigma or otherwise noted and were of the highest grade commercially available.

### Example 1

1.1 Aminoethylcysteine Modification and Protease Digestion of Peptides and Proteins

For model peptides, approximately 100 μg peptide was dissolved in 50 μL of a 4:3:1 solution of H₂O:DMSO:EtOH. 23 μl of a sat. Ba(OH)₂ solution and 1 μL of 5M NaOH were added, and the reaction was incubated at room temperature. After 1 hour, 50 μL of a 1M solution of cysteamine in H₂O was added directly to this reaction and the reaction was incubated an additional hour at room temperature. Reactions were analyzed by diluting into 1 mL H₂0/0.1% TFA and separating the reaction products by reverse phase HPLC (Rainin SD-200 system equipped with a Zorbax 300 C-18 9.4 mm×25 cm column). Individual fractions were analyzed by electrospray MS offline using a Waters Micromass ZQ.

For site-mapping, modified peptides were reconstituted in either 10 mM Tris, pH 8.5 (Trypsin) or 10 mM Tris, pH 8.5, 1 mM EDTA (Lys-C) and digested overnight at 37° C. Reactions were desalted (by C₁₈ ZipTip or HPLC) and analyzed by electrospray MS. For FRET monitoring of the Lys-C digestion of diastereomeric aminoethylcysteine peptides, peptide 6 diastereomers (˜5 μg) were separated by HPLC, and digested with 5 μg Lys-C. Reaction progress was monitored as emission at 420 nm following excitation at 320 nm in a Molecular Devices SpectraMax GeminiXS fluorescence plate reader as described (Meldal and Breddam, Anal Biochem, 1991. 195(1): p. 141-7).

α-Casein was pretreated with performic acid for 2 hours to quantitatively oxidize cysteine residues (Oda et al., Nat Biotechnol, 2001, 19(4): 379-82); β-casein, which contains no cysteine residues, was used as provided by the manufacturer. Proteins were modified using the same conditions as described for peptides. Following aminoethylcysteine modification, proteins were transferred to the appropriate buffer by gel filtration (PD-10, Amersham). Sequential digests with Trypsin and then Lys-C were carried out at 37° C. for approximately 6 hours.

For exclusive phosphorylation site cleavage, the MARCKS substrate was dissolved in 100 mM NaHCO₃, pH 8.5 and treated with approximately 100 equivalents of sulphosuccinimidyl acetate (Pierce) for 2 hours at room temperature to quantitative acetylate lysine residues. This reactions was desalted by HPLC and subjected to aminoethylcysteine modification and digestion. Crude reactions were desalted by C₁₈ ZipTip and analyzed.

1.2 Results

A panel of six synthetic phosphoserine peptides and five phosphothreonine peptides was derivatized. It was discovered that the β-elimination conditions typically reported (Oda et al., Nat Biotechnol, 2001: 19(4), 379-82; Goshe et al., Anal Chem, 2001, 73(11): 2578-86; Jaffe et al., Biochemistry, 1998: 37(46), 16211-24 (˜1M hydroxide, 42-55° C.) can yield extensive peptide degradation. By using a lower hydroxide concentration (˜150 μM), limiting reactions to one hour at room temperature, and including barium as a specific catalyst for phosphate elimination (Byford, Biochem J, 1991, 280 (Pt 1): 261-5; Adamczyk et al., Rapid Commun Mass Spectrom, 2001, 15(16): 1481-8), it was possible to achieve nearly quantitative β-elimination for all peptides tested. Addition of cysteamine directly to this reaction cleanly converted the intermediate alkene containing residues to aminoethylcysteine or β-methyl aminoethylcysteine under mild conditions. Finally, digestion of these modified peptides with Lys-C liberated peptide fragments corresponding to selective cleavage at the site of phosphorylation, allowing for unambiguous identification of the phosphorylation sites from the exact masses of the fragments (Table 1).

TABLE 1

Exp. Mass (Calcd. Mass)

Lys-C

| | | | |
|---|---|---|---|
| Sequence | Dehydroalanine | Aminoethylcys | Digest |

| | | | |
|---|---|---|---|
| GRTGRRNpSIH | 1476.4 | 1554.6 | 610.4 |
| DIL | (1476.8) | (1553.8) | (610.4) |
| DLDVPIPGRFD | 2094.0 | 2170.6 | 1801.0 |
| RRVpSVAAE | (2094.1) | (2171.1) | (1801.0) |
| SLRRSpSC*FG | 3141.4 | 3218.2 | 2472.8 |
| GRIDRIGAQSG | (3141.5) | (3218.5) | (2473.1) |
| LGC*NSFRY | | | |
| KRPpSQRHGSK | 1325.4 | 1402.2 | 712.6 |
| Y | (1324.7) | (1401.8) | (712.4) |
| LRRApSLG | 754.6 | 831.6 | 661.4 |
| | (754.4) | (831.5) | (661.4) |
| ZFRPpSGFY*D | 1134.7 | 1211.7 | 684.6 |
| | (1134.5) | (1211.5) | (684.3) |
| ZFRPpTGFY*D | 1147.5 | 1224.5 | 697.4 |
| | (1147.5) | (1224.5) | (697.3) |
| KRpTIRR | 810.6 | 887.6 | n/a |
| | (810.5) | (887.6) | |

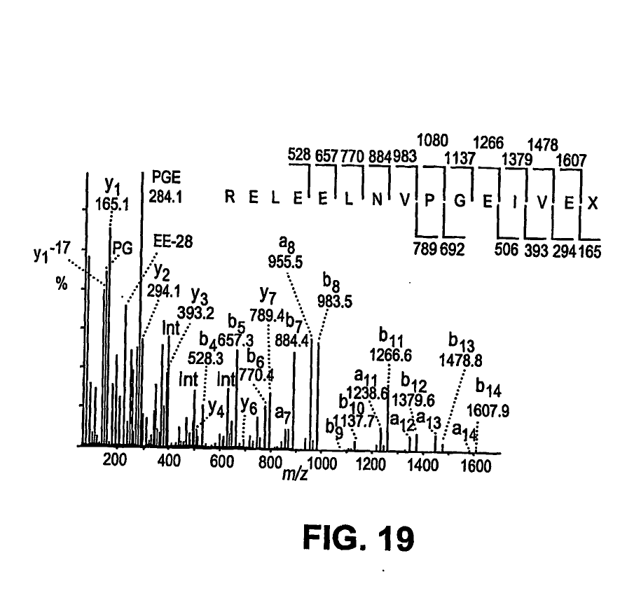

The strategy was extended to facilitate mapping phosphorylation sites on full length proteins. Two proteins (α- and β-casein) were selected that contain three and five sites of phosphorylation, respectively, and each protein was subjected to aminoethylcysteine modification followed by co-digestion with trypsin and Lys-C. One pmole of protein from each digest was separated by nanoflow liquid chromatography and analyzed inline using a QSTAR quadrapole orthogonal time of flight mass spectrometer. From the exact mass data, we were able to identify eight peptides corresponding to direct cleavage at all eight of the known phosphorylation sites of the two proteins (Table 2). The identity of each peptide was independently confirmed by LC-MS/MS sequencing, and we found that the aminoethylcysteine modified residues fragment normally upon collision induced dissociation (CID), generating readily interpretable tandem mass spectra (FIG. 19A). An interesting feature of the MS/MS spectra of aminoethylcysteine digest products was the presence of a unique y₁ ion at 165.1 Da (FIG. 19A). This mass signature is generated by loss of a C-terminal aminoethylcysteine residue and is distinct from the fragmentation of any naturally occurring amino acid. It may be possible to exploit this unique fragmentation using a precursor ion scanning strategy in order to selectively analyze ions that have undergone aminoethylcysteine modification.

TABLE 2

| | |
|---|---|
| Exp. | Calcd. |

| | | | | |
|---|---|---|---|---|
| Protein | Residues | Peptide Sequence | Mass | Mass |

| | | | | |
|---|---|---|---|---|
| αₛ₁-casein | 43-58 | DIGK*EK*TEDQAM(SO₂)EDIK | 1917.75 | 1917.79 |
| αₛ₁-casein | 47-58 | (K*)EK*TEDQAM(SO₂)EDIK | 1486.54 | 1486.60 |
| αₛ₁-casein | 49-58 | (K*)TEDQAM(SO₂)EDIK | 1211.47 | 1211.51 |
| αₛ₁-casein | 106-119 | VPQLEIVPNK*AEER | 1639.85 | 1639.85 |
| αₛ₁-casein | 106-115 | VPQLEIVPNK* | 1154.59 | 1154.62 |
| αₛ₁-casein | 153-164 | TVDMEK*TEVFTK | 1477.61 | 1477.67 |
| αₛ₁-casein | 159-164 | (K*)TEVFTK | 724.37 | 724.39 |
| αₛ₁-casein | 1-25 | RELEELNVPGEIVEK*LK*K*K*E | 3038.40 | 3038.48 |
| | | ESITR | | |
| β-casein | 1-19 | RELEELNVPGEIVEK*LK*K*K* | 2323.12 | 2323.13 |
| β-casein | 1-18 | RELEELNVPGEIVEK*LK*K* | 2177.04 | 2177.08 |
| β-casein | 1-17 | RELEELNVPGEIVEK*LK* | 2030.93 | 2031.02 |
| β-casein | 1-15 | RELEELNVPGEIVEK* | 1771.79 | 1771.89 |
| β-casein | 33-48 | FQK*EEQQQTEDELQDK | 2040.81 | 2040.88 |
| β-casein | 36-48 | (K*)EEQQQTEDELQDK | 1619.66 | 1619.70 |
| MARCKS | 1-25 | ac-KKKKKRFK*FKKK*FKLSGFK | | |
| | | *FKKNKK | | |
| MARCKS | 1-19 | ac-KKKKKRFK*FKKK*FKLSGFK | | |
| | | * | | |
| MARCKS | 1-12 | ac-KKKKKRFK*FKKK* | | |
| MARCKS | 1-8 | ac-KKKKKRFK* | | |
| MARCKS | 9-25 | FKKK*FKLSGFK*FKKNKK | | |
| MARCKS | 9-19 | FKKK*FKLSGFK* | | |
| MARCKS | 9-12 | FKKK* | | |
| MARCKS | 13-25 | FKLSGFK*FKKNKK | | |
| MARCKS | 13-19 | FKLSGFK* | | |
| MARCKS | 20-25 | (K*)FKKNKK | | |

The symbol K* represents aminoethylcysteine.

One surprising feature of the MS data was the detection of phosphopeptides predicted to be in low abundance in the casein digests. Two peptides were observed (the aminoethylcysteine modified and the corresponding digest product, Table 2) corresponding to a phosphorylation site on α₂-casein. The identification of α₂-casein phosphopeptides in casein digests is typically not reported, likely because that protein is a minor component of commercial casein preparations (which are predominantly α₁-casein). Thus, the aminoethylcysteine modification enhances the mass spectrometric response of formerly phosphorylated peptides (Steen et al., J Am Soc Mass Spectrom, 2002, 13(8): 996-1003 (due to replacement of the acidic phosphoserine with the basic aminoethylcysteine), such that low abundance phosphopeptides may be detected without enrichment. Consistent with this view, detection was possible of aminoethylcysteine modified phosphopeptides from α-casein during analysis of β-casein digests; α-casein is known to be a low level (<5%) contaminant in commercial β-casein preparations (Goshe et al., Anal Chem, 2002, 74(3): 607-16). FIG. 13, panel A shows enhanced sensitivity using the aminoethylcysteine modification relative to phosphoserine in panel B for the β-casein digest. FIG. 14, panel E shows detection by MALDI-MS at a level of 25 fmol in an unfractionated trypsin digest (panel A illustrates the mass spectra at 500 fmol of chemically modified peptide, panel B illustrates the mass spectra at 250 fmol of chemically modified peptide, panel C illustrates the mass spectra at 125 fmol of chemically modified peptide, and panel D illustrates the mass spectra at 250 fmol of chemically modified peptide).

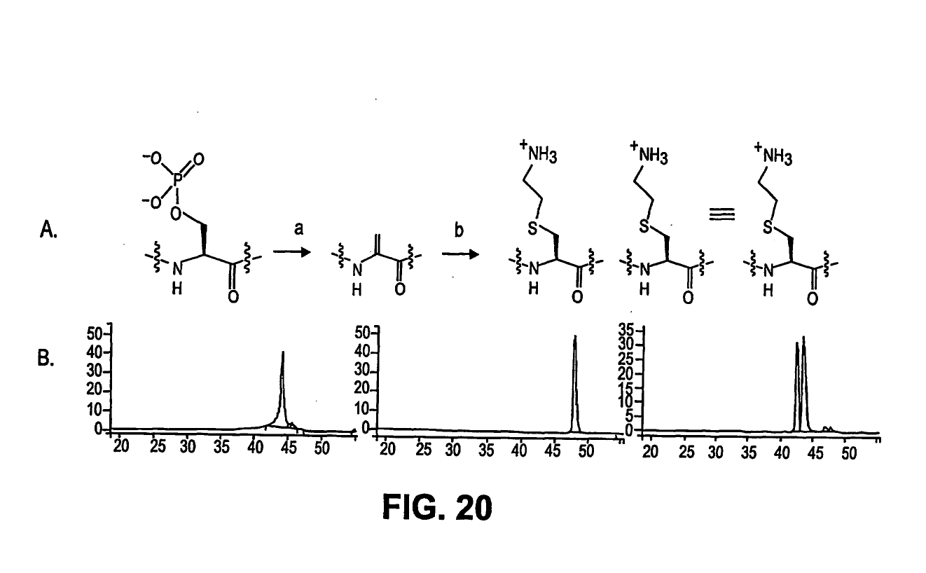

During aminoethylcysteine modification, non-stereoselective nucleophilic attack occurs at Cβ of the phosphorylated amino acid, generating two diastereomeric ainioethylcysteine peptides (D and L) in an approximately 1:1 mixture (FIG. 20B). We have confirmed that peptides containing the L stereochemistry at Cα are substrates for trypsin, whereas those with the D stereochemistry are not (FIG. 20C). As a consequence, under conditions where proteolysis is allowed to proceed to completion, cleavage occurs at approximately 50% of the sites for any given phosphopeptide. Thus, the resultant mass spectrum contains peaks for both the intact (derivatized) phosphopeptide, as well as the fragments generated from cleavage at the phosphorylation site (Table 2). For a single tryptic peptide containing multiple phosphorylation sites, this partial digestion generates a “ladder” of peptides corresponding to successive single cleavage at each of the phosphorylation sites. This effect is illustrated dramatically for β-casein peptide₁₋₂₅, where 4 phosphorylation sites are found in a 5 amino acid sequence. In this case, we identify 5 unique peptides corresponding to sequential cleavage at each of those sites (Table 2). In practice, this intrinsic partial digestion should be advantageous for phosphopeptide mapping, by providing additional mass information and thereby simplifying database searching to identify the correct peptide.

In some cases, it may be desirable to obtain cleavage exclusively at the phosphorylation site (not at lysine residues), generating larger fragments that provide information about the gross topology of phosphorylation. For example, the coexistence of unique phosphoisoforms of a single peptide (variants of a single protein that contain distinct combinations of phosphorylated residues) can be investigated by this type of digestion. The structure of such phosphoisoforms is challenging to probe by traditional methods, since trypsin digestion intrinsically disconnects information about phosphorylation sites that are separated by more than 10 to 20 residues (the frequency of a lysine or arginine residue). Alternatively, cleavage exclusively at phosphorylation sites would facilitate phosphorylation mapping by N-terminal Edman degradation, since the first residues sequenced would be those directly C-terminal to the site of phosphorylation.

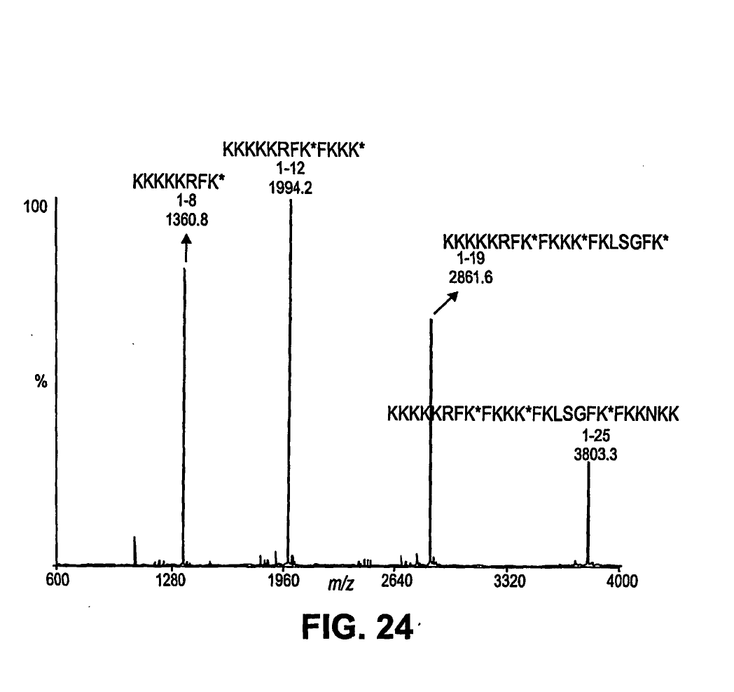

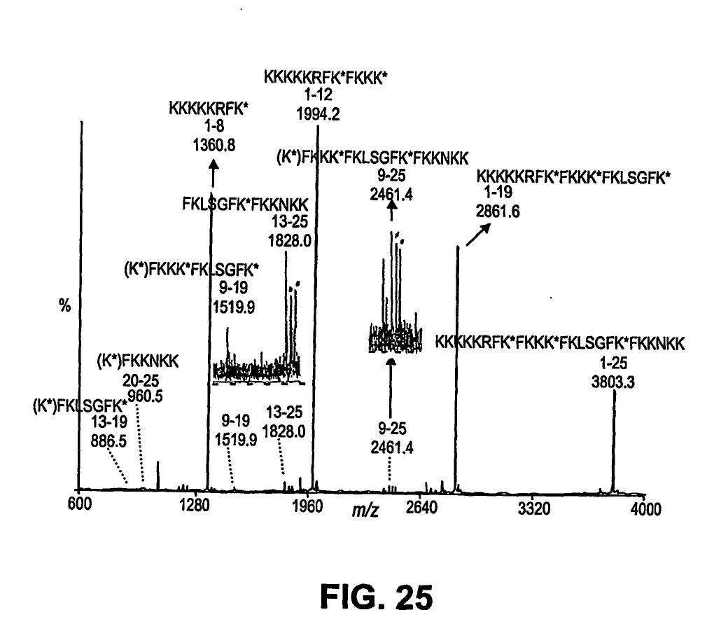

To achieve exclusive cleavage at phosphorylation sites, sulfosuccinimidyl acetate is used to first block all of the lysine residues on substrate peptides or proteins. The MARCKS substrate—a 25 residue peptide containing 12 lysines and three phosphoserine residues—was chosen to test this approach. This sequence was selected because the density of lysine residues should provide a significant test of our ability to achieve phosphorylation-selective cleavage. The MARCKS substrate was acetylated, modified as aminoethylcysteine, digested with Lys-C, and finally subjected to MS analysis by MALDI. The MALDI spectrum from this digest exhibits four prominent peaks corresponding to the three N-terminal fragments generated by successive cleavage at each of the three phosphorylation sites, plus the intact (undigested) peptide (FIG. 24). Six additional peaks were detected corresponding to the fragments that would be predicted to result from every combination of cleavage at the three phosphorylation sites (FIG. 25). The identity of each peptide fragment was independently confirmed by LC-MS/MS. Minimal cleavage was detected at the acetylated lysine residues, although a more significant degree of cleavage was observed at an arginine residue (fragment mH+=1067) upon extended incubation with high concentrations of Lys-C.

For guanidination reactions, the MARCKS substrate or β-casein was dissolved in 0.5 M O-methylisourea, pH 10.5 and incubated overnight at 37° C. essentially as described in Beardsley et al., Anal. Chem. 74: 1884-1890 (2002), Brancia et al., Rapid Commun. Mass Spectrom. 14: 2070-2073 (2000), and Bonetto et al., Anal. Chem. 69: 1315-1319 (1997). For acetylation reactions, the MARCKS substrate was dissolved in 100 mM NaHCO3, pH 8.5 and treated with approximately 100 equivalents of sulphosuccinimidyl acetate (Pierce) for 2 hours at room temperature to quantitatively acetylate lysine residues. Reactions were desalted by BPLC or dialysis and subjected to aminoethylcysteine modification and Lys-C digestion as above.

Mass spectra were obtained by matrix-assisted laser desorption ionization time-of-flight (MALDI-TOF) mass spectrometry on a Voyager DESTR plus (Applied Biosystems). All mass spectra were acquired in positive-ionization mode with reflectron optics. The instrument was equipped with a 337 nm nitrogen laser and operated under delayed extraction conditions in reflectron mode; a delay time of 190 nsec, and grid voltage 66-70% of full acceleration voltage (20-25 kV). For linear mode experiments, the delay time was 100 nsec and the grid voltage 93.4% of the acceleration voltage. Prior to MALDI-MS analysis, the proteolytic reaction mixtures were desalted with reversed-phase Zip TipsC18 (C-18 resin, Millipore). All peptide samples were prepared using a matrix solution consisting of 33 mM HCCA in acetonitrile/methanol (1/1; v/v); 1 μL of analyte (0.1-1 pmol of material) was mixed with 1 μL of matrix solution, and then air-dried at room temperature on a stainless steel target. Typically, 50 laser shots were used to record each spectrum. The obtained mass spectra were externally calibrated with an equimolar mixture of angiotensin I, ACTH 1-17, ACTH 18-39, and ACTH 7-38.

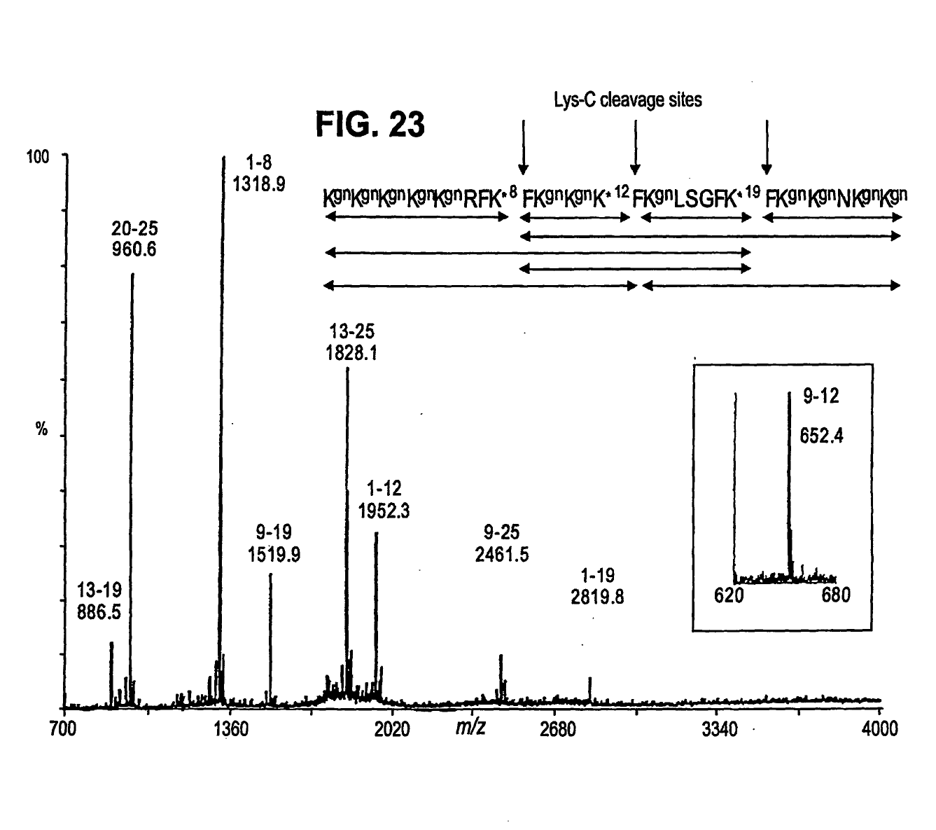

As shown in FIG. 23, guanidination of lysine residues facilitates Lyc-C cleavage exclusively at aminoethylcysteine residues. In addition, as shown in FIG. 24, acetylation of lysine residues facilitates Lyc-C cleavage exclusively at aminoethylcysteine residues, with a different hierarchy of ion intensities.

### Example 2

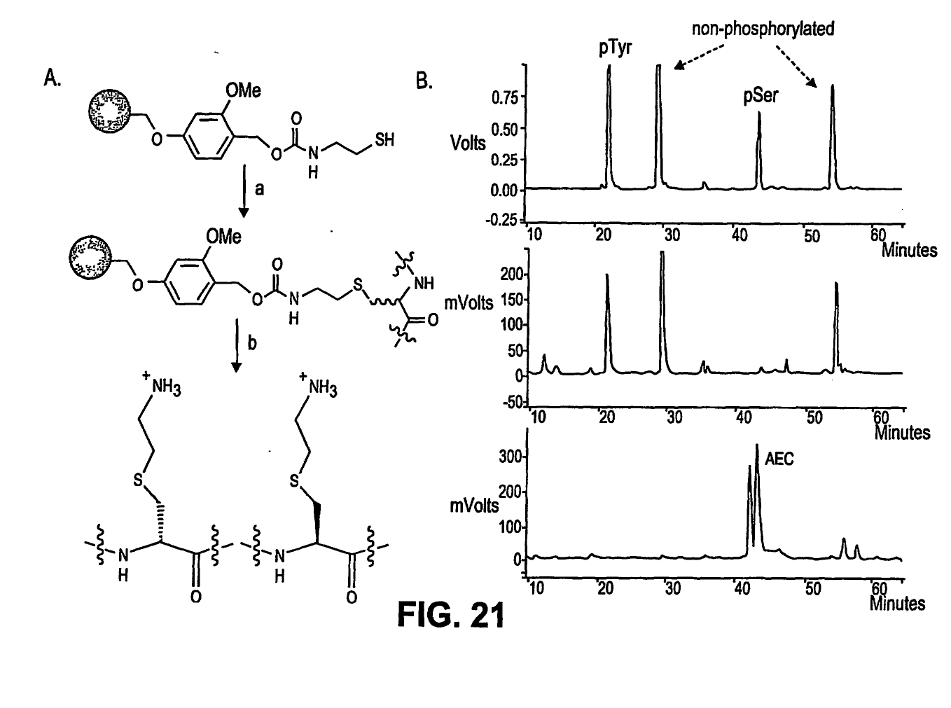

To prepare the solid phase reagent, a polyethyleneglycol-polystyrene (PEG-PS) copolymer base resin (TentaGel AC) was loaded with cysteamine as the benzyl carbamate (FIG. 21A). This reagent was designed to incorporate two important features that facilitate aminoethylcysteine modification. First, a PEG-PS resin was selected, which swells in both organic and aqueous solvents, so that the resin capture can be performed in one pot under conditions optimized and validated for the solution phase chemistry. Secondly, the methoxybenzyl carbamate linkage is stable to the basic conditions of the β-elimination reaction, allowing for efficient peptide capture, but highly acid labile, facilitating aminoethylcysteine peptide release by brief treatment with trifluoroacetic acid (TFA). The use of this solid phase reagent facilitates automation (Zhou et al., Nat Biotechnol, 2002, 20(5): 512-5) and offer advantages over similar approaches that rely on selective biotinylation, which has been observed to complicate MS spectra (Oda et al., Nat Biotechnol, 2001. 19(4): 379-82).

2.1 Resin Synthesis

Resin was loaded using a modification of the procedure of Dorff (Dorff, et al., Tetrahedron Letters, 1995, 36(10): 1589-1592). Briefly, Tentagel AC resin (5 g) was swelled in anhydrous THF (75 ml) at room temperature under an inert atmosphere. 2.5 g of 1,1 carbonyldiimidazole was added and the resulting mixture was stirred for 3 hours. The resin was filtered, washed with THF, Et₂O, and dried in vacuo overnight.

Before use, 5 g cysteamine HCl salt was dissolved in 45 ml H₂O, the pH was adjusted to 12 with NaOH, and the cysteamine was extracted with CH₂Cl₂. The organic phase was dried with MgSO₄, filtered, and the solvent removed in vacuo to give a clear oil. Approximately 1 g of this oil was added 2 g of the activated resin swelled in THF (25 ml). 2 mL N-methylmorpholine was added and the resin was heated to 60° C. for 4-6 hours under an inert atmosphere.

The resin was filtered, washed with THF, Et₂O, dried in vacuo, and stored at −20° C. Immediately before use, the resin was deprotected by brief treatment (15 min.) with 100 mM DTT in H₂O to expose the cysteamine thiol. Quantification of resin loading with Ellman's reagent typically yielded 70-80% loading (0.20 to 0.25 mmol/g).

### Example 3

Although phosphorylation is the most common post-translational modification, phosphoproteins are often present at low abundance and phosphorylated sub-stoichiometrically, making genome wide phosphorylation analysis an analytical challenge. A method for phosphopeptide purification as well as phosphorylation site mapping would greatly facilitate this process. For this purpose, an exemplary reaction of the invention was adapted to the solid phase, so that modification and enrichment of phosphopeptides or proteins can occur in one step (FIG. 21A).

3.1 Solid-Phase Capture and Modification of Phosphoserine Peptides

Following deprotection, the resin was washed with 5 times with H₂O and 5 times with 4:3:1H₂O:DMSO:EtOH. Peptides were dissolved in 250 μL of 4:3:1 H₂O:DMSO:EtOH and added to 80 mg of resin swelled in the same. 225 μL of sat. Ba(OH)₂ and 10 μL of 5M NaOH were added and the reaction was incubated for one hour at room temperature. After one hour, the resin was rinsed successively with H₂O, DMF, CH₂Cl₂ and Et₂O and dried overnight in vacuo. To release the peptides, the dried resin was suspended in 1 ml of 95:2.5:2.5 TFA:Me₂S:H₂0 for 15 minutes at room temperature. The resin was then filtered, washed 3 times with 1 mL TFA, and the filtrate was concentrated in vacuo. The released peptides were taken up in H₂O/0.1% TFA and analyzed by HPLC and MS as described.

3.2 Results

The ability of the reagent from Example 2 to capture phosphopeptides and release them as the aminoethylcysteine derivative was tested. Two non-phosphorylated peptides, one phosphotyrosine peptide, and one phosphoserine peptide were added to the resin as an approximately equimolar mixture (FIG. 21B, panel 1). After incubation with the resin under β-elimination conditions for one hour, the flow-through was analyzed by HPLC; the non-phosphorylated and phosphotyrosine peptides were detected intact, but the phosphoserine peak was absent, consistent with selective capture of the phosphoserine peptide (FIG. 21B, panel 2). Brief treatment with 95% TFA released the two diastereomeric aminoethylcysteine peptides (FIG. 21B, panel 3).

The present invention provides a novel method for mapping post-translational modifications of peptides, e.g. phosphorylation, methylation, and glycosylation, and a solid-phase material for converting a modified amino acid into a substrate for an enzyme. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### Example 4

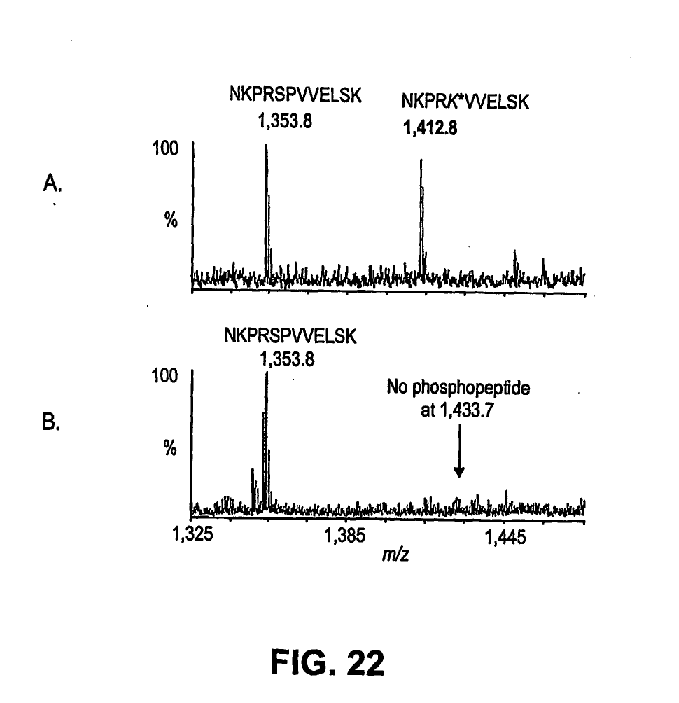

N-terminal His6-tagged GRK2 was expressed in SF9 insect cells and purified using Ni-NTA beads (Qiagen) as described 37. Bovine tubulin was a gift from Ron Vale. Tubulin (5 μM) and GRK2 (0.6 μM) were incubated in 100 μl of 20 mM HEPES, pH 7.4, 2.0 mM EDTA, 10 mM MgCl₂ containing 1 mM ATP. Kinase reactions were performed at 25° C. for 3 hours 22, after which the reactions were desalted by microdialysis, subjected to aminoethylcysteine modification, digested with either Lys-C/Trypsin or Lys-C/Asp-N, and finally analyzed by LC-MS/MS and MALDI-MS as described above. In a similar fashion, purified GRK2 (˜5 μg) was subjected to aminoethylcysteine modification, digested with Lys-C, and then analyzed by mass spectrometry as described. Table 3 shows the identification of serine and threonine phosphorylation sites in GRK2 and tubulin using the cysteamine chemical modification reagent. FIG. 22 illustrates the enhancement of the mass spectroscopy response of a GRK2 phosphorylation site using the cysteamine chemical modification reagent (panel A) versus no chemical modification reagent (Panel B).

TABLE 3

| | |
|---|---|
| EXP. | CALC. |

| | | | | |
|---|---|---|---|---|
| PROTEIN | RESIDUES | SEQUENCE | MASS | MASS |

| | | | | |
|---|---|---|---|---|
| GRK2 | 666-677 | NKPRK*PVVPELSK | 1411.78 | 1411.80 |
| GRK2 | 668-677 | PRK*PVVPELSK | 1169.62 | 1169.66 |
| GRK2 | 671-677 | K*PVVPELSK | 771.40 | 771.45 |
| β-Tubulin | 404-416 | DEMEFK^{T}*EASNMN | 1604.52 | 1604.58 |
| β-Tubulin | 404-416 | DEM**EFK^{T}*EASNMN | 1620.60 | 1620.57 |
| β-Tubulin | 404-409 | DEMEFK^{T}* | 829.34 | 829.30 |
| β-Tubulin | 404-409 | DEM**EFK^{T}* | 845.38 | 845.30 |
| β-Tubulin | 417-426 | DLVK*EYQQYQ | 1330.51 | 1330.58 |
| β-Tubulin | 421-426 | K*EYQQYQ | 857.32 | 857.36 |
| β-Tubulin | 417-420 | DLVK* | 491.26 | 491.24 |

The symbol M** represents methionine sulfoxide. The symbols K* and K^{T}* represent, aminoethylcysteine and β-methyl aminoethylcysteine, respectively.

What is claimed is:

1. A method of mapping the location of a post-translational modification of a post-translationally modified peptide, said method comprising:
(a) contacting said peptide with a chemical modification reagent that converts a post-translationally modified amino acid residue of said peptide into a substrate for a peptidase, thereby producing a chemically modified peptide comprising a chemically modified amino acid residue;
(b) contacting said chemically modified peptide with said peptidase under conditions appropriate to degrade said chemically modified peptide, thereby producing a degraded chemically modified peptide; and
(c) querying said degraded chemically modified peptide to ascertain said location of said post-translational modification.

2. The method of claim 1, further comprising:
(d) prior to step (a), contacting a substrate amino acid of said peptide that is a natural substrate for said peptidase with a blocking agent thereby converting said substrate amino acid into a side-chain protected amino acid that is not a substrate for said peptidase.

3. The method of claim 2, wherein said substrate amino acid is a lysine, wherein said blocking agent converts said lysine into a side-chain protected lysine selected from the group consisting of a carbamate, an amide, an N-sulfonyl, an N-sulfenyl, an N-nitro, an N-nitroso, an N-oxide, an imine, an N-alkyl amine, an N-aryl amine, an N-phosphinyl, an N-phosphoryl, and an enamine.

4. The method of claim 2, wherein said side chain protected lysine is selected from the group consisting of Lys(Aloc), Lys(Ac), Lys(Boc), Lys(biotinyl), Lys(2-bromo-Z), Lys(2-chloro-Z), Lys(Dnp), Lys(Fmoc), Lys(For), Lys(Me)₂, Lys(nicatinoyl), Lys(Tfa), Lys(Tos), Lys(Z), Lys(Z)(isopropyl), Lys(Boc)(isopropyl), Lys(dansyl), Lys(Dde), Lys(Me)₃, Lys(Mtt), Lys(palitoyl, Lys(TNM), Lys(acetimidoyl), Lys(2,4,-dichloro-Z), Lys(Me), Lys(p-nitro-Z), Lys(5/6 FAM), Lys(pyrenebutyryl), and Lys(guanidinyl).

5. The method of claim 1, wherein said substrate amino acid is aspartic acid, wherein said blocking agent converts said aspartic acid into a side-chain protected aspartic acid selected from an ester, an amide, an oxalose, an oxazolines, a stannyl ester, and an hydrazide.

6. The method of claim 1, wherein side chain protected aspartic acid is selected from the group consisting of Asp(OBzl), Asp(OcHex), Asp(OtBu), Asp(OMpe), Asp(Ofm), Asp(Osu), Asp(2-phenyisopropyl ester), and Asp(ONp).

7. The method of claim 1, wherein said peptidase is selected from the group consisting of a serine endopeptidase, a metalloendopeptidase, a cysteine endopeptidase, and an aspartic endopeptidase.

8. The method of claim 1, wherein said peptidase is a lysine-specific peptidase.

9. The method of claim 8, wherein said lysine-specific peptidase is selected from the group consisting of endoproteinase Lys-C, lysyl endopeptidase, trypsin, plasma kallikrein, oligopeptidase B, tryptase, plasmin, acrosin, granzyme A, yapsin 1, peptidyl-Lys metalloendopeptidase, and magnolsyin.

10. The method of claim 8, wherein said lysine-specific peptidase is selected from the group consisting of endoproteinase Lys-C, lysyl endopeptidase and trypsin.

11. The method of claim 1, wherein said peptidase is an aspartate-specific peptidase.

12. The method of claim 11, wherein said aspartate-specific peptidase is selected from peptidyl-aspartate metalloendopeptidase and nepenthesin.

13. The method of claim 1, wherein said querying comprises mass spectrographic detection of said chemically modified amino acid residue of said degraded chemically modified peptide.

14. The method according to claim 1, further comprising:
(e) prior to step (a), contacting said peptide with an elimination reagent that causes the elimination of a post-translationally added substituent of said post-translationally modified amino acid residue.

15. The method of claim 14, wherein said post-translationally modified amino acid residue is selected from the group consisting of a post-translationally modified serine and a post-translationally modified threonine.

16. The method of claim 14, wherein said post-translationally modified amino acid residue is a phosphorylated amino acid residue.

17. The method according to claim 14, wherein said elimination is a elimination giving rise to an alkene moiety.

18. The method according to claim 1, wherein said modification reagent reacts with said post-translationally modified amino acid residue via a Michael addition.

19. The method of claim 18, wherein said modification reagent is selected from the group consisting of sodium sulfate and cysteamine.

20. A reactive solid phase material comprising:
(a) a solid support; and
(b) a solid support reactive moiety immobilized on said solid support, wherein said solid support reactive moiety is reactive towards a synthetically modified amino acid residue of a post-translationally modified peptide, said synthetically modified amino acid residue produced by elimination a post-translationally added substituent of said post-translationally modified peptide.

21. The material according to claim 20, wherein said synthetically modified amino acid residue comprises an alkene moiety.

22. A method of immobilizing a post-translationally modified peptide comprising a post-translationally modified amino acid, said method comprising:
(i) contacting said peptide with an elimination reagent that causes the elimination of a post-translationally added substituent of said post-translationally modified amino acid residue thereby producing a synthetically modified amino acid;
(ii) reacting said synthetically modified amino acid with a reactive solid phase material thereby immobilizing said post-translationally modified peptide, said reactive solid phase material comprising:
(a) a solid support; and
(b) a solid support reactive moiety immobilized on said solid support, wherein said solid support reactive moiety is reactive towards said synthetically modified amino acid residue.


SEQUENCE LISTING (57 sequences)

SEQ ID NO: 1 - protein, 7 aa, Artificial Sequence
  Peptide containing a cysteic acid residue
LRRAXLG

SEQ ID NO: 2 - protein, 4 aa, Artificial Sequence
  Digested peptide fragment after cleavage with peptidyl-aspartate metalloendopeptidase (Asp-N)
LRRA

SEQ ID NO: 3 - protein, 9 aa, Artificial Sequence
  Undigested betamethyl aminoethylcysteine modified peptide
ZFRPXGFXE

SEQ ID NO: 4 - protein, 9 aa, Artificial Sequence
  Undigested phosphorylated peptide
ZFRPXGFXE

SEQ ID NO: 5 - protein, 13 aa, Artificial Sequence
  Peptide containing an aminoethylcysteine modification
NKPPRXPVVELSK

SEQ ID NO: 6 - protein, 15 aa, Artificial Sequence
  Peptide containing a phosphoserine
NKKPPRXSPVVELSK

SEQ ID NO: 7 - protein, 13 aa, Artificial Sequence
  unmodified peptide
NKPPRSPVVELSK

SEQ ID NO: 8 - protein, 23 aa, Artificial Sequence
  Guanidinated MARCKS peptide
XXXXXRFXFXXXFXLSGFXXNXX

SEQ ID NO: 9 - protein, 8 aa, Artificial Sequence
  Acetylated MARCKS peptide after cleavage with endoproteinase Lys-C (Lys-C)
KKKKKRFX

SEQ ID NO: 10 - protein, 12 aa, Artificial Sequence
  Acetylated MARCKS peptide after cleavage with endoproteinase Lys-C (Lys-C)
KKKKKRFXFKKX

SEQ ID NO: 11 - protein, 19 aa, Artificial Sequence
  Acetylated MARCKS peptide after cleavage with endoproteinase Lys-C (Lys-C)
KKKKKRFXFKKXFKLSGFX

SEQ ID NO: 12 - protein, 25 aa, Artificial Sequence
  Acetylated MARCKS peptide after cleavage with endoproteinase Lys-C (Lys-C)
KKKKKRFXFKKXFKLSGFXFKKNKK

SEQ ID NO: 13 - protein, 8 aa, Artificial Sequence
  Acetylated MARCKS peptide after cleavage with endoproteinase Lys-C (Lys-C)
XFKLSGFX

SEQ ID NO: 14 - protein, 7 aa, Artificial Sequence
  Acetylated MARCKS peptide after cleavage with endoproteinase Lys-C (Lys-C)
XFKKNKK

SEQ ID NO: 15 - protein, 12 aa, Artificial Sequence
  Acetylated MARCKS peptide after cleavage with endoproteinase Lys-C (Lys-C)
XFKKXFKLSGFX

SEQ ID NO: 16 - protein, 13 aa, Artificial Sequence
  Acetylated MARCKS peptide after cleavage with endoproteinase Lys-C (Lys-C)
FKLSGFXFKKNKK

SEQ ID NO: 17 - protein, 18 aa, Artificial Sequence
  Acetylated MARCKS peptide after cleavage with endoproteinase Lys-C (Lys-C)
XFKKXFKLSGFXFKKNKK

SEQ ID NO: 18 - protein, 13 aa, Artificial Sequence
  Synthetic phosphoserine peptide
GRTGRRNXIHDIL

SEQ ID NO: 19 - protein, 19 aa, Artificial Sequence
  Synthetic phosphoserine peptide
DLDVPIPGRFDRRVXVAAE

SEQ ID NO: 20 - protein, 28 aa, Artificial Sequence
  Synthetic phosphoserine peptide
SLRRSXCFGGRIDRIGAQSGLGCNSFRY

SEQ ID NO: 21 - protein, 11 aa, Artificial Sequence
  Synthetic phosphoserine peptide
KRPXQRHGSKY

SEQ ID NO: 22 - protein, 7 aa, Artificial Sequence
  Synthetic phosphoserine peptide
LRRAXLG

SEQ ID NO: 23 - protein, 9 aa, Artificial Sequence
  Synthetic phosphoserine peptide
ZFRPXGFYD

SEQ ID NO: 24 - protein, 9 aa, Artificial Sequence
  Synthetic phosphotyrosine peptide
ZFRPXGFYD

SEQ ID NO: 25 - protein, 6 aa, Artificial Sequence
  Synthetic phosphotyrosine peptide
KRXIRR

SEQ ID NO: 26 - protein, 16 aa, Artificial Sequence
  Alpha-s1 casein peptide from co-digestion with trypsin and endoproteinase Lys-C (Lys-C)
DIGXEXTEDQAXEDIK

SEQ ID NO: 27 - protein, 13 aa, Artificial Sequence
  Alpha-s1 casein peptide from co-digestion with trypsin and endoproteinase Lys-C (Lys-C)
XEXTEDQAXEDIK

SEQ ID NO: 28 - protein, 11 aa, Artificial Sequence
  Alpha-s1 casein peptide from co-digestion with trypsin and endoproteinase Lys-C (Lys-C)
XTEDQAXEDIK

SEQ ID NO: 29 - protein, 14 aa, Artificial Sequence
  Alpha-s1 casein peptide from co-digestion with trypsin and endoproteinase Lys-C (Lys-C)
VPQLEIVPNXAEER

SEQ ID NO: 30 - protein, 10 aa, Artificial Sequence
  Alpha-s1 casein peptide from co-digestion with trypsin and endoproteinase Lys-C (Lys-C)
VPQLEIVPNX

SEQ ID NO: 31 - protein, 12 aa, Artificial Sequence
  Alpha-s2 casein peptide from co-digestion with trypsin and endoproteinase Lys-C (Lys-C)
TVDMEXTEVFTK

SEQ ID NO: 32 - protein, 7 aa, Artificial Sequence
  Alpha-s2 casein peptide from co-digestion with trypsin and endoproteinase Lys-C (Lys-C)
XTEVFTK

SEQ ID NO: 33 - protein, 25 aa, Artificial Sequence
  Beta casein peptide from co-digestion with trypsin and endoproteinase Lys-C (Lys-C)
RELEELNVPGEIVEXLXXXEESITR

SEQ ID NO: 34 - protein, 19 aa, Artificial Sequence
  Beta casein peptide from co-digestion with trypsin and endoproteinase Lys-C (Lys-C)
RELEELNVPGEIVEXLXXX

SEQ ID NO: 35 - protein, 18 aa, Artificial Sequence
  Beta casein peptide from co-digestion with trypsin and endoproteinase Lys-C (Lys-C)
RELEELNVPGEIVEXLXX

SEQ ID NO: 36 - protein, 17 aa, Artificial Sequence
  Beta casein peptide from co-digestion with trypsin and endoproteinase Lys-C (Lys-C)
RELEELNVPGEIVEXLX

SEQ ID NO: 37 - protein, 15 aa, Artificial Sequence
  Beta casein peptide from co-digestion with trypsin and endoproteinase Lys-C (Lys-C)
RELEELNVPGEIVEX

SEQ ID NO: 38 - protein, 16 aa, Artificial Sequence
  Beta casein peptide from co-digestion with trypsin and endoproteinase Lys-C (Lys-C)
FQXEEQQQTEDELQDK

SEQ ID NO: 39 - protein, 14 aa, Artificial Sequence
  Beta casein peptide from co-digestion with trypsin and endoproteinase Lys-C (Lys-C)
XEEQQQTEDELQDK

SEQ ID NO: 40 - protein, 25 aa, Artificial Sequence
  MARCKS peptide from co-digestion with trypsin and endoproteinase Lys-C (Lys-C)
XKKKKRFXFKKXFKLSGFXFKKNKK

SEQ ID NO: 41 - protein, 19 aa, Artificial Sequence
  MARCKS peptide from co-digestion with trypsin and endoproteinase Lys-C (Lys-C)
XKKKKRFXFKKXFKLSGFX

SEQ ID NO: 42 - protein, 12 aa, Artificial Sequence
  MARCKS peptide from co-digestion with trypsin and endoproteinase Lys-C (Lys-C)
XKKKKRFXFKKX

SEQ ID NO: 43 - protein, 8 aa, Artificial Sequence
  MARCKS peptide from co-digestion with trypsin and endoproteinase Lys-C (Lys-C)
XKKKKRFX

SEQ ID NO: 44 - protein, 17 aa, Artificial Sequence
  MARCKS peptide from co-digestion with trypsin and endoproteinase Lys-C (Lys-C)
FKKXFKLSGFXFKKNKK

SEQ ID NO: 45 - protein, 11 aa, Artificial Sequence
  MARCKS peptide from co-digestion with trypsin and endoproteinase Lys-C (Lys-C)
FKKXFKLSGFX

SEQ ID NO: 46 - protein, 7 aa, Artificial Sequence
  MARCKS peptide from co-digestion with trypsin and endoproteinase Lys-C (Lys-C)
FKLSGFX

SEQ ID NO: 47 - protein, 7 aa, Artificial Sequence
  MARCKS peptide from co-digestion with trypsin and endoproteinase Lys-C (Lys-C)
XFKKNKK

SEQ ID NO: 48 - protein, 6 aa, Artificial Sequence
  N-terminal His6-tag
HHHHHH

SEQ ID NO: 49 - protein, 13 aa, Artificial Sequence
  GRK2 peptide from digestion with endoproteinase Lys-C (Lys-C)
NKPRXPVVPELSK

SEQ ID NO: 50 - protein, 11 aa, Artificial Sequence
  GRK2 peptide from digestion with endoproteinase Lys-C (Lys-C)
PRXPVVPELSK

SEQ ID NO: 51 - protein, 9 aa, Artificial Sequence
  GRK2 peptide from digestion with endoproteinase Lys-C (Lys-C)
XPVVPELSK

SEQ ID NO: 52 - protein, 12 aa, Artificial Sequence
  Beta-tubulin peptide from digestion with endoproteinase Lys-C (Lys-C)/Asp-N codigest
DEMEFXEASNMN

SEQ ID NO: 53 - protein, 12 aa, Artificial Sequence
  Beta-tubulin peptide from digestion with endoproteinase Lys-C (Lys-C)/Asp-N codigest
DEXEFXEASNMN

SEQ ID NO: 54 - protein, 6 aa, Artificial Sequence
  Beta-tubulin peptide from digestion with endoproteinase Lys-C (Lys-C)/Asp-N codigest
DEMEFX

SEQ ID NO: 55 - protein, 6 aa, Artificial Sequence
  Beta-tubulin peptide from digestion with endoproteinase Lys-C (Lys-C)/Asp-N codigest
DEXEFX

SEQ ID NO: 56 - protein, 10 aa, Artificial Sequence
  Beta-tubulin peptide from digestion with endoproteinase Lys-C (Lys-C)/Asp-N codigest
DLVXEYQQYQ

SEQ ID NO: 57 - protein, 7 aa, Artificial Sequence
  Beta-tubulin peptide from digestion with endoproteinase Lys-C (Lys-C)/Asp-N codigest
XEYQQYQ